(19) United States
(12) Patent Application Publication
(10) Pub. No.: US 2026/0043042 A1
(43) Pub. Date: Feb. 12, 2026

(54) Plants with Modified Deoxyhypusine Synthase Genes

(71) Applicant: AGRIBODY TECHNOLOGIES, INC., Torance, CA (US)

(72) Inventor: Jerald S. Feitelson, Torrance, CA (US)

(73) Assignee: AGRIBODY TECHNOLOGIES, INC., (US)

(21) Appl. No.: 18/881,220

(22) PCT Filed: Jul. 6, 2023
(86) PCT No.: PCT/US2023/069698

§ 371 Date: Jan. 3, 2025

Related U.S. Application Data
(60) Provisional application No. 63/367,759, filed on Jul. 6, 2022.

(51) Int. Cl. C12N 15/82; C12N 9/10

(52) U.S. Cl. CPC C12N 15/8279; C12N 9/1085; C12N 15/8262; C12N 15/8273; C12Y 205/01046

(57) ABSTRACT

The disclosure relates to methods of producing a plant with delayed senescence comprising at least one nucleotide deletion, insertion, or substitution into at least one copy of a gene encoding deoxyhypusine synthase (DHS) in the plant, wherein the nucleotide deletion, insertion, or substitution decreases the activity of DHS encoded by the gene in the plant. The disclosure also relates to plants produced by the methods described herein and progeny thereof.

### CROSS REFERENCE TO RELATED APPLICATIONS

This application claims priority to U.S. Provisional Patent Application No. 63/367,759, filed Jul. 6, 2022, the contents of which are hereby incorporated by reference herein, in their entireties, for all purposes.

### REFERENCE TO SEQUENCE LISTING SUBMITTED ELECTRONICALLY

This application contains a Sequence Listing in XML format submitted electronically herewith via EFS-Web. The contents of the XML copy, created on Jul. 6, 2023 is named “SequenceListing” and is 374,680 bytes in size. The Sequence Listing is incorporated herein by reference in its entirety.

### BACKGROUND

Senescence is the terminal phase of biological development in the life of a plant. It presages death and occurs at various levels of biological organization including the whole plant, organs, flowers and fruit, tissues and individual cells.

The onset of senescence can be induced by different factors both internal and external. Senescence is a complex, highly regulated developmental stage in the life of a plant or plant tissue, such as fruit, flowers and leaves. Senescence results in the coordinated breakdown of cell membranes and macromolecules and the subsequent mobilization of metabolites to other parts of the plant.

In addition to the programmed senescence which takes place during normal plant development, death of cells and tissues and ensuing remobilization of metabolites occurs as a coordinated response to external, environmental factors. External factors that induce premature initiation of senescence, which is also referred to as necrosis or apoptosis, include environmental stresses such as temperature, drought, poor light or nutrient supply, as well as pathogen attack. Plant tissues exposed to environmental stress also produce ethylene, commonly known as stress ethylene (Buchanan-Wollaston (1997) J. Exp. Botany 48:181-199; Wright, M. (1974) Plant 120:63-69). Ethylene is known to cause senescence in some plants.

Senescence is not a passive process, but, rather, is an actively regulated process that involves coordinated expression of specific genes. During senescence, the levels of total RNA decrease and the expression of many genes is switched off (Bate et al. (1991) J. Exper. Botany 42:801-11; Hensel et al. (1993) The Plant Cell 5:553-64). However, there is increasing evidence that the senescence process depends on de novo transcription of nuclear genes. For example, senescence is blocked by inhibitors of mRNA and protein synthesis and enucleation. Molecular studies using cDNA from senescing leaves and green leaves for in vitro translation experiments show a changed pattern of leaf protein products in senescing leaves (Thomas et al. (1992) J. Plant Physiol. 139:403-12). With the use of differential screening and subtractive hybridization techniques, many cDNA clones representing senescence-induced genes have been identified from a range of different plants, including both monocots and dicots, such as Arabidopsis, maize, cucumber, asparagus, tomato, rice and potato. Identification of genes that are expressed specifically during senescence is hard evidence of the requirement for de novo transcription for senescence to proceed.

The events that take place during senescence appear to be highly coordinated to allow maximum use of the cellular components before necrosis and death occur. Complex interactions involving the perception of specific signals and the induction of cascades of gene expression must occur to regulate this process. Expression of genes encoding senescence related proteins is probably regulated via common activator proteins that are, in turn, activated directly or indirectly by hormonal signals. Little is known about the mechanisms involved in the initial signaling or subsequent co-ordination of the process.

Coordinated gene expression requires factors involved in transcription and translation, including initiation factors. Translation initiation factor genes have been isolated and characterized in a variety of organisms, including plants. Translation initiation factors can control the rate at which mRNA populations are moved out of the nucleus, the rate at which they are associated with a ribosome and to some extent can affect the stability of specific mRNAs. (Zuk et al. (1998) EMBO J. 17:2914-2925). Indeed, one such translation initiation factor, which is not required for global translation activity, is believed to shuttle specific subsets of mRNAs from the nucleus to the cytoplasm for translation (Jao et al. (2002) J. Cell. Biochem. 86:590-600; Wang et al. (2001) J. Biol. Chem. 276:17541-17549; Rosorius et al. (1999) J. Cell Sci. 112:2369-2380). This translation factor is known as the eukaryotic initiation factor 5A (eIF-5A), and is the only protein known to contain the amino acid hypusine (Park et al. (1988) J. Biol. Chem. 263:15264-15269).

Eukaryotic translation initiation factor 5A (eIF-5A) is an essential protein factor approximately 17 kDa in size, which is involved in the initiation of eukaryotic cellular protein synthesis. It is characterized by the presence of hypusine [N-(4-amino-2-hydroxybutyl)lysine], a unique modified amino acid and known to be present only in eIF-5A. Hypusine is formed post-translationally via the transfer and hydroxylation of the butylamine group from the polyamine, spermidine, to the side chain amino group of a specific lysine residue in eIF-5A. Activation of eIF-5A involves transfer of the butylamine residue of spermidine to the lysine of eIF-5A, forming hypusine and activating eIF-5A. In eukaryotes, deoxyhypusine synthase (DHS) mediates the post-translational synthesis of hypusine in eIF-5A. The hypusine modification has been shown to be essential for eIF-5A activity in vitro using a methionyl-puromycin assay.

Hypusine is formed on eIF-5A post-translationally through the conversion of a conserved lysine residue by the action of deoxyhypusine synthase (DHS; EC 1.1.1.249) and deoxyhypusine hydroxylase (DOHH; EC 1.14.99.29). DHS cDNA has been directly sequenced or predicted from genomic sequences in dozens of plant species, including Arabidopsis thaliana (GenBank Accession No. NM_120674), alfalfa (U.S. Pat. No. 8,563,285), banana (GenBank Accession No. XM_009405857), camelina (GenBank Accession No. XP_010452500), canola (GenBank Accession No. XM_013859772), carnation (GenBank Accession No. AF296080), cocoa (GenBank Accession No. CGD0006914), coffee (GenBank Accession No. GR986281), soybean (GenBank Accession No. BM092515), tobacco (GenBank Accession No. NM_001325620), tomato (GenBank Accession No. NM_001247566), wheat (GenBank Accession No. FJ376389), and many others. DOHH cDNA sequences have also been identified in some plants, including Medicago truncatula (GenBank Accession No. XM_013594404).

DHS converts a conserved lysine residue of eIF-5A to deoxyhypusine through the addition of a butylamine group derived from spermidine. This intermediate form of eIF-5A is then hydroxylated by DHH to become hypusine (Park et al. (1997) Biol. Signals 6:115-123). Both the deoxyhypusine and the hypusine form of eIF-5A are able to bind cDNA in vitro (Liu et al. (1997) Biol. Signals 6:166-174). Although the function of eIF-5A is not fully understood, there is some evidence that it may regulate cell division (Park et al. (1998) J. Biol. Chem. 263:15264-15269; Tome et al. (1997) Biol. Signals 6:150-156), and senescence. (Wang et al. (2001) J. Biol. Chem. 276:17541-17549). It appears that several organisms are known to have more than one isoform of eIF-5A, which would suit the premise that each isoform is a specific shuttle to specific suites of mRNAs that are involved in such processes as cell division and senescence.

Wang et al. demonstrated that an increased level of DHS cDNA correlates with fruit softening and natural and stress-induced leaf senescence of tomato (Wang et al. (2001) J. Biol. Chem. 276:17541-17549; (2003) Plant Molecular Biology 52: 1223-1235; and (2005) Plant Physiology 138:1372-1382). Furthermore, when the expression of DHS was suppressed in transgenic tomato plants by introducing a DHS antisense cDNA fragment under the regulation of a constitutive promoter, the tomato fruit from these transgenic plants exhibited dramatically delayed senescence as evidenced by delayed fruit softening and spoilage. See U.S. Pat. Nos. 6,878,860, 6,900,368, 7,070,997, and 7,226,784. Since DHS is known to activate eIF-5A, these data suggest that the hypusine-modified eIF-5A (active eIF-5A) may regulate senescence through selective translation of mRNA species required for senescence. This is further demonstrated through the down-regulation of DHS in Arabidopsis thaliana (“AT”) by antisense of the full length or 3′UTR cDNA under the control of a constitutive promoter. By down regulating Arabidopsis thaliana DHS (“AT-DHS”) expression and making it less available for eIF-5A activation, senescence was delayed by approximately 2 weeks (See Duguay et al. (2007) Journal of Plant Physiology 164:408-420 & U.S. Pat. No. 7,226,784]. Not only was senescence delayed, but also an increase in seed yield, an increase in stress tolerance and an increase in biomass were observed in the transgenic plants, where the extent of each phenotype was determined by the extent of the down-regulation of DHS.

Although down-regulation of DHS in plants by means of antisense transgenic plants is expected to generate plants with advantageous agronomic properties, such as resistance to stress, delayed senescence, and increased yields, transgenic plants in general have several disadvantages. Creation of transgenic DHS plants requires the introduction of foreign DNA, including the antisense gene, which often use viral promoters for strong expression, as well as selection genes. Furthermore, viral promoters are often recognized by the plant and turned-off, leading to loss of antisense expression in future generations of transgenic plants. A better strategy for down-regulation of DHS in plants, for instance alfalfa, is to use genome editing to modify the gene in order reduce or eliminate the activity of the translated DHS protein. Arabidopsis thaliana and many other plants only have one copy of the DHS gene per haploid genome, as shown by Southern blot (Wang et al. (2001) J. Biol. Chem. 276:17541-17549) and full genome sequencing. Since alfalfa is a tetraploid plant, using a genome editing technique could disrupt in separate progeny from the same experiment: one out of the four DHS copies found in its genome thereby reducing DHS activity in the plant tissues by approximately 25%, two out of the four DHS copies found in its genome thereby reducing activity by approximately 50%, or three out of the four DHS copies found in its genome thereby reducing activity by approximately 75%. Screening independent progeny expressing each of these residual activity levels could lead to identification of clones that demonstrate the maximum degree of improved resistance to stress and delayed senescence via incomplete hypusination of eIF-5A isoforms involved in stress and senescence pathways. It is unlikely to find progeny that have all four DHS copies disrupted since this should be a lethal event, given that homozygous knockout of DHS has been demonstrated to be lethal in mice and yeast (Templin et al. (2011) Cell Cycle 10:1043-9; Sasaki et al. (1996) FEBS Lett. 384:151-4).

Genome editing makes use of various technologies to manipulate the genome of either plants or animals by inserting, deleting, or substituting specific genetic sequences in a highly specific manner. Many genome editing methods exist, and include, but are not limited to, use of transgenic DNA sequences flanked by sequences homologous to the intended site of modification (homologous recombination), or methods using engineered nucleases, including Meganucleases, zinc finger nucleases, (ZFNs), transcription activator-like effector-based nucleases (TALEN), ARC nuclease (ARCUS), and CRISPR-based systems using Cas9, CRISPR-Cpf1, CRISPR-Cms1, or many others. In all these systems, nucleases create site-specific double-stranded DNA breaks which are then repaired via homologous recombination or nonhomologous end-joining to create the targeted mutation.

An example of homologous recombination is the rapid trait development system (RTDS) (Beetham et al. (1999) Proc. Natl. Acad. Sci. USA 96:8774-8778; Kochevenko and Willmitzer (2003) Plant Physiol. 132:174-184). RTDS uses a Gene Repair Oligonucleotide (GRON) to introduce a mismatch error into the sequence of a targeted gene in a highly specific manner. This mismatch is then repaired by the plant's natural DNA repair system that uses the GRON as a template in order to create the desired modification.

The clustered regularly interspaced short palindromic repeats (CRISPR)-Cas9 genome editing system has been used in a wide variety of organisms, including monocot and dicot plants. One or more single guide RNA (sgRNA), with a target sequence homologous to the desired gene being targeted for editing, is introduced along with Cas9 nuclease, and are used to direct the Cas9 protein to a specific genomic site (reviewed by Ma and Liu (2016) Curr. Protoc. Mol. Biol, DOI: 10.1002/cpmb.10]. Similar experiments can be designed using other double stranded nucleases, such as Cms1 (Begemann and Gray and U.S. Pat. No. 9,896,696, the contents of which are hereby incorporated by reference herein, in their entireties).

Transcription activator-like effectors (TALE) are naturally occurring transcription effectors that use a simple code of tandem repeats that allows for customizable generation of TALEs that recognize a DNA sequence with high specificity. When combined with a functional domain, such as FokI nuclease (TALEN), targeted gene disruption is possible. Binding of the TALEN nuclease to its engineered recognition sequence results in double-stranded DNA break and subsequent recruitment of the non-homologous end joining repair machinery, resulting in either small deletions or insertions that lead to disrupted gene function. TALEN has been used to modify economically important food crops and biofuels and is being explored in correcting genetic errors that underpin certain human diseases. TALEN can also be combined with the simultaneous introduction of a targeting segment of DNA that contains homology to the cleavage site in order to specifically introduce the targeted sequences into the locus using homologous recombination.

Meganucleases (also called homing endonucleases) are nucleases that recognize large sequences (12-40 base pairs) that occur very rarely, and ideally only once, in the genome (Porteus et al. (2005) Nat. Biotechnol. 23:967-73]. Meganucleases normally recognize palindromic sequences, however, by producing a pair of monomers which recognize two different half-sites which, as heterodimers, form a meganuclease that cleaves a non-palindromic site. ARCUS is a genome editing technology that is based on ARC nuclease, a totally synthetic homing endonuclease-like enzyme that is derived from a naturally occurring homing endonuclease. ARC nuclease can be customized to recognize a specific DNA sequence, allowing a precise DNA break, often at only a single site in the genome. This DNA break allows genome modification, including insertions, deletions, or substitutions, by homologous recombination.

The function of the DHS enzyme is well understood. The deoxyhypusine synthase reaction catalyzed by DHS involves interaction with three different substrates: spermidine, NAD⁺, and eIF-5A precursor protein (eIF-5A(Lys)]. The first step of the reaction is the NAD-dependent dehydrogenation of spermidine, the second step involves trans-imination to form the DHS-imine intermediate, the third step involves trans-imination for the eIF-5A-imine intermediate, and the fourth step is the enzyme-coupled reduction of the eIF-5A-imine intermediate (Joe et al. (1997) J. Biol. Chem. 272:32679-685]. The enzyme-imine intermediate bond is formed between the 4-amino-butyl moiety of spermidine and the F-amino group of K329 in the human enzyme [Joe et al. J. Biol. Chem. (1997) 272:32679-685]. Upon addition of the eIF-5A(Lys) precursor, the butylamine group is transferred to K50 of human eIF-5A and then reduced to form deoxyhypusine. K329 of human DHS is therefore crucial for DHS enzymatic activity, since it is absolutely required for the transfer of the butylamine group from spermidine to eIF-5A.

A large number of amino acid residues that have been shown to be critical for DHS function were disclosed in US Patent Application Publication US2019/0203220A1, the contents of which are hereby incorporated by reference herein, in their entireties. This application focuses on a heretofore unknown, hypervariable small region of the protein that can be deleted in a homozygous state while maintaining viability. Indeed, the present invention is based, in whole or part, on a homozygous in-frame deletion of this small region that reduces activity of the DHS protein to produce highly desirable traits in plants.

The amino acid sequence alignment of the many plant DHS proteins shows many long regions of nearly perfect homology, but the 6 amino acid region herein described as the “hypervariable region” has very little identity between plant species as shown by the boxed region of sequences in FIG. 1C; and is a small region of “disordered residues” incapable of being crystallized as shown in FIG. 2B.

There is a large and growing body of scientific research studying the possible function of “intrinsically disordered protein regions,” including specificity of transcription factors, proteins involved in formation of subcellular condensates, and human diseases such as cancer, cardiovascular disease, and many neurodegenerative disorders

(Brodsky, S., et al. (2021) Curr Opin Struct Biol 71:110-115; Borcherds, W., et al. (2021) Curr. Opin. Struct. Biol. 67:41-50; Garaizar, A., et al. (2020) Molecules 25:4705; Santofimia-Castaño, et al. (2020) Cell Mol. Life Sci. 77:1695-1707; Kulkarni, P & V. Uversky (2019) Biomolecules 9:147; Uversky, V. (2015) Front. Aging Neurosci. 7:1-6, the contents of which are hereby incorporated by reference herein, in their entireties.

It is likely that the hypervariable region of the DHS protein is involved in the formation of subcellular protein complexes, including its direct substrate eIF-5A. Indeed, one proposed mechanism of action of the hypusination of eIF-5A is the selective export from the nucleus to the cytoplasm of RNAs involved in senescence/apoptosis. Thus, small deletions of residues in the hypervariable region of DHS could result in less effective protein complex formations, with consequent reductions in senescence. The accompanying phenotypic traits could include extended shelf life, enhanced tolerance to abiotic stress, some tolerance to necrotrophic pathogens, and greater yields.

The ability to construct homozygous mutations that reduce but don't eliminate catalytic function of the DHS protein is particularly important in crops grown from hybrid seeds. Examples include corn, wheat, grain sorghum, cotton, peanuts and many other crops. In these cases, elite inbred strains are used as parents that are homozygous at most loci. One or both parental lines having reduced activity due to targeted mutations in their DHS genes would be particularly advantageous in the resulting hybrid seeds sold to farmers.

Presently, there is no widely applicable method for controlling the onset of programmed cell death (including senescence) caused by either internal or external, e.g., environmental stress, factors. It is, therefore, of interest to develop senescence modulating technologies that are applicable to all types of plants and that are effective at the earliest stages in the cascade of events leading to senescence. Genome editing of DHS is a possible solution to reduce loss in plant yields due to environmental stress, as well as increase shelf life of perishable produce such as fruits, vegetables, and flowers.

### SUMMARY

In some aspects and embodiments, the present disclosure provides protein sequences of deoxyhypusine synthase (DHS) from plant species, notably tomato, and the polynucleotides that encode these proteins, including mRNA and genomic sequences.

In some embodiments, the present disclosure also relates to methods involving genome editing involving either deletions, insertions, or substitutions to disrupt the activity of these DHS proteins by targeting amino acid residues in the hypervariable region.

In some embodiments, the present disclosure provides a method for genome editing of plants to control the onset of senescence, either age-related senescence or environmental stress-induced senescence. One of several genome editing technologies, including but not limited to RTDS, TALEN, ARCUS or CRISPR is used to introduce a deletion, insertion, or substitution in the region of an amino acid residue in the hypervariable region, in order to lead to the reduction of DHS protein activity, thereby reducing the level of functionally active endogenous senescence-induced DHS protein, and reducing and/or preventing activation of eIF-5A and ensuing downstream expression of the genes that mediate senescence.

In some embodiments, the methods of the present disclosure provide genome-edited plants are generated and monitored for growth, development and either natural or delayed senescence. Plants or detached parts of plants (e.g., cuttings, flowers, vegetables, fruits, seeds or leaves) exhibiting prolonged life or shelf life, (e.g., extended life of flowers, reduced fruit or vegetable spoilage), enhanced biomass, increased seed yield, increased resistance to physiological disease (e.g., blossom end rot, reduced seed aging and/or reduced yellowing of leaves) due to reduction in the level of senescence-induced DHS are selected as desired products having improved properties including reduced leaf yellowing, reduced petal abscission, reduced fruit and vegetable spoilage during shipping and storage. These superior plants are propagated. Similarly, plants exhibiting increased resistance to environmental stress (e.g., high or low temperatures, drought, low nutrient levels, high salt, crowding, pathogen infection, and/or physiological disease) are selected as superior products.

In some embodiments, the species of plant which may be used in the methods of the invention is not limited to and includes, e.g., ethylene-sensitive and ethylene-insensitive plants; fruit bearing plants such as apricots, apples, oranges, bananas, grapefruit, pears, tomatoes, strawberries, avocados, grapes, etc. In some embodiments, the plant is a vegetable such as carrots, peas, lettuce, cabbage, turnips, potatoes, broccoli, asparagus, peppers, zucchini, beans, etc.

In some embodiments, the plant is a flower such as roses, carnations, mums, orchids, etc.

In some embodiments, the plant is an agronomic crop plant species such as corn, rice, soybean, alfalfa, wheat, cotton, sugarbeet, canola, Camelina, sorghum, sunflower, cassava, peanuts, and includes forest plants such as poplar, other trees and the like. In general, any plant that can take up DNA molecules for genome editing can be used in the methods of the invention and may include plants of a variety of ploidy levels, including haploid, diploid, tetraploid and polyploid. The plant may be either a monocotyledon or dicotyledon, and may use C3 or C4 photosynthesis.

### BRIEF DESCRIPTION OF THE DRAWINGS

FIG. 1A-FIG. 1I. Alignment of human DHS protein and plant DHS proteins. (! is either of L, Q, I or V, $ is either of L or M, % is either of R, F or Y, # is any of H, T, K, N, A, D, Q or E). The hypervariable region comprising six amino acids from E105 to D110 (human DHS numbering), equivalent to six amino acids from H76 to E81 (tomato DHS numbering), is highlighted in FIG. 1C.

FIG. 2A-FIG. 2B. 3-Dimensional ribbon drawing of the topology of the human DHS monomer and (b) Schematic diagram of secondary structure of the DHS monomer showing the hypervariable region in red oval, between the a3 alpha helix and b1 beta sheet, here numbered between amino acids 74 and 96. This figure, adapted from [Liao et al. (1998) Structure 6:23-32], suggests this small region consists of “disordered residues.”

FIG. 3. Transformed T₀ TF2465 DHS1_4 tomato plant with chimeric leaves. The dark green sectors indicated by arrows contain a homozygous deletion in the hypervariable region that removed 6 nucleotides resulting in loss of 2 residues of the HELPTE domain, converting it to HEPE and the darker green leaf phenotype.

FIG. 4A-FIG. 4B. Schematic of genomic DNA of DHS genes from various plant species. Boxes represent exons. Lines represent introns. Thick blue lines represent the hypervariable regions described in this application. Red lines and down arrows (↓) represent the lysine residues that forms a covalent intermediate with a butylamine moiety. Pink lines and asterisks (*) represent the active site.

FIG. 5. DHS1 model of genomic sequence and relative position of the gRNAs in tomato. Boxes represent exons, dashed lines represent introns. Thin vertical yellow lines represent 3 of the major mutagenic targets for genome editing, with a focus on the hypervariable region using guide G1_75.

FIG. 6. Structure of the Agrobacterium tumifaciens vector 11,503 bp insert between the left border (LB) and right border (RB) carrying the NptII selectable marker driven by the Arabidopsis thaliana Ubq10 promoter, the dicot codon-optimized Cas9 protein, the DHS1-G1 guide RNA, and the Citrine gene as a visible marker.

FIG. 7. Summary of in-frame mutations in T1 tomato plants.

FIG. 8. Sequence analysis of a DHS1_4 T2 TF2465 tomato plant with a bialellic D6 in-frame deletion mutation (-TGCCCA) (SEQ ID NO:156) in the hypervariable region.

FIG. 9. Sequence analysis of a DHS1_73 T2 TF2465 tomato plant with a bialellic D3 in-frame deletion mutation (-CGG) (SEQ ID NO:157) in the hypervariable region.

FIG. 10. Sequence analysis of a DHS1_135 T2 TF4415 tomato plant with a bialellic D3 in-frame deletion mutation (-CAC) (SEQ ID NO:158) in the hypervariable region.

FIG. 11. Sequence analysis of a DHS1_158 T2 TF4145 tomato plant with a bialellic D15 in-frame deletion mutation (-CGGAGGATTGCAGTG) (SEQ ID NO:159) in the hypervariable region.

FIG. 12. Photographs of DHS1_135 T2 TF4145 tomato fruit with a bialellic D3 in-frame deletion mutation (-CAC) in the hypervariable region at harvest, 2 weeks after harvest, 4 weeks after harvest, and 5 weeks after harvest.

### DETAILED DESCRIPTION

Unless defined otherwise, all technical and scientific terms used herein have the same meaning as is commonly understood by one of skill in the art to which this disclosure belongs. All patents and publications referred to herein are incorporated by reference in their entireties.

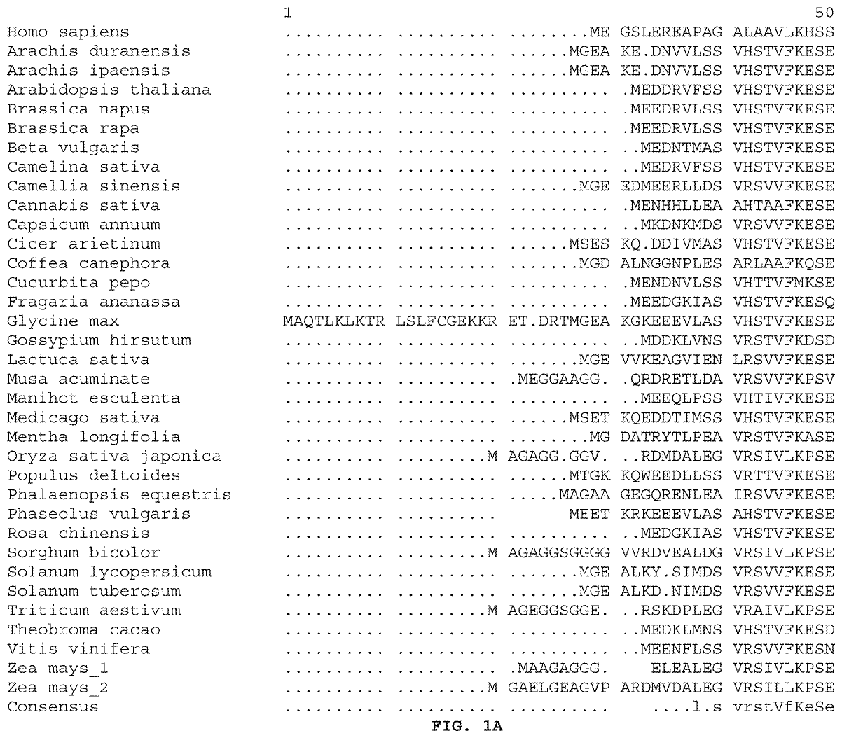

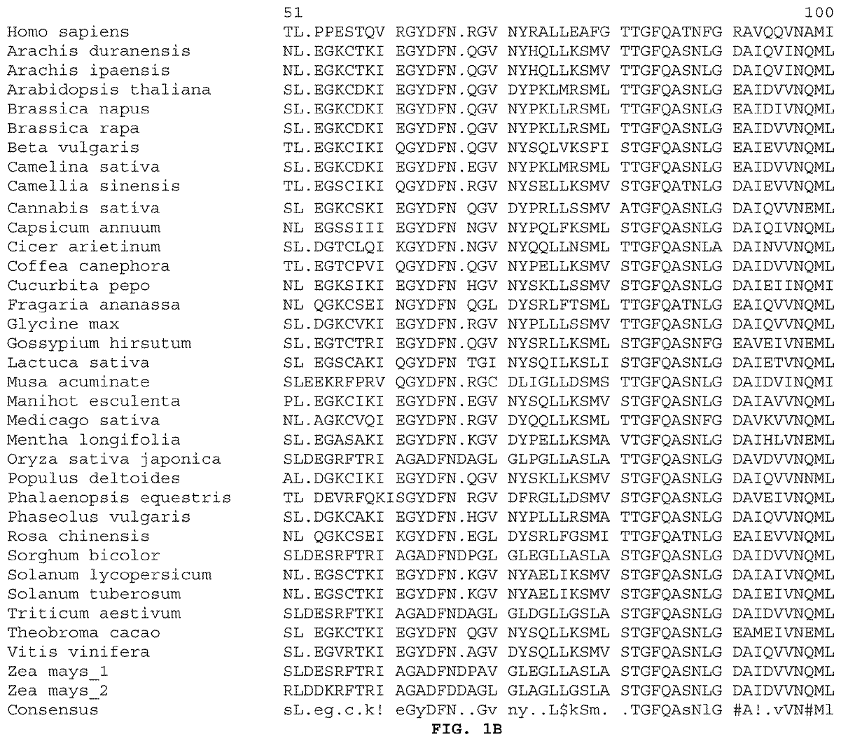

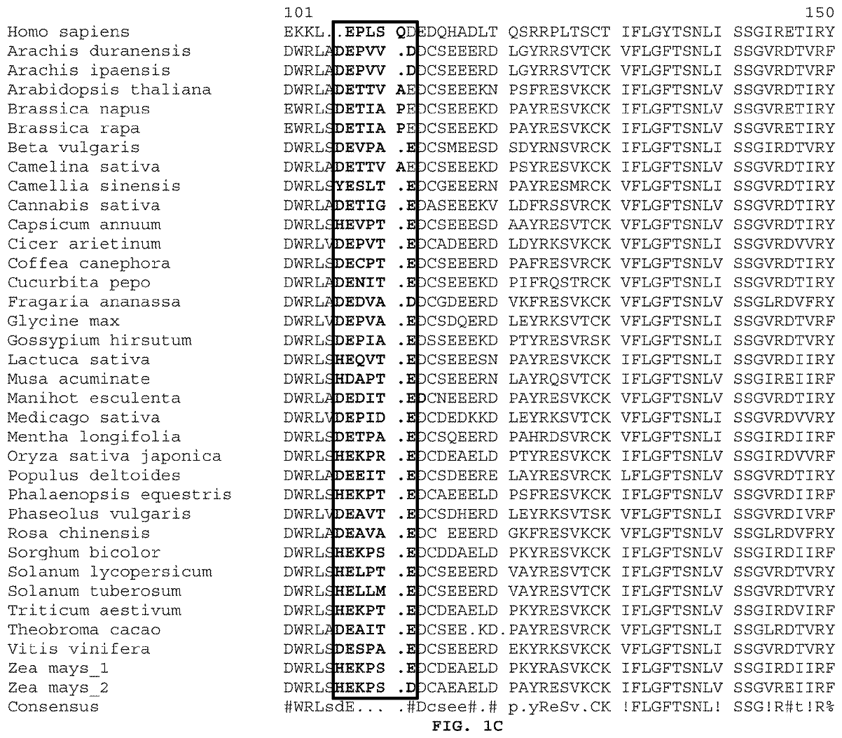

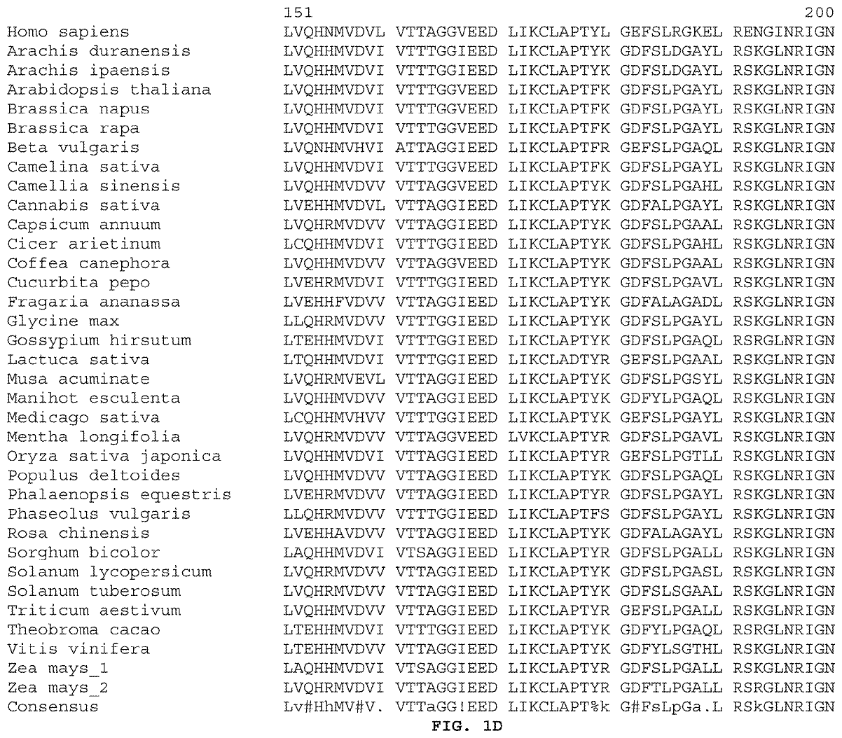

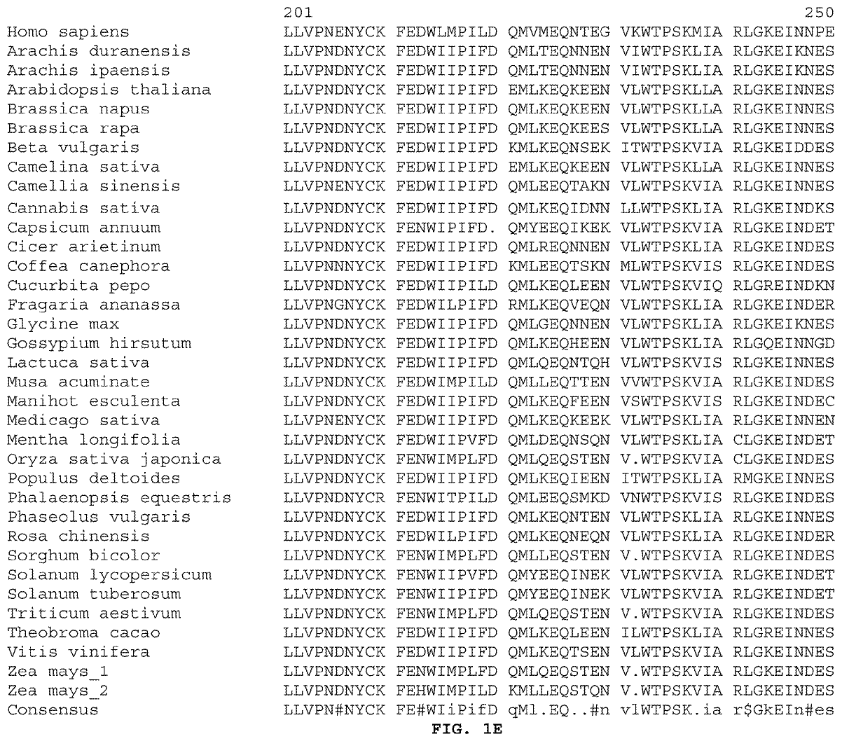

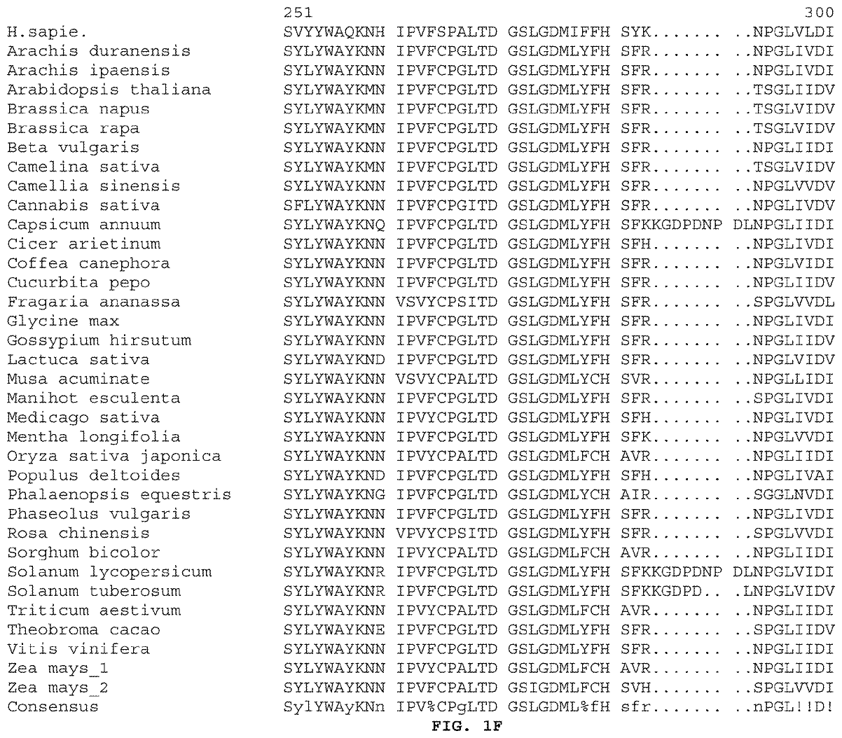

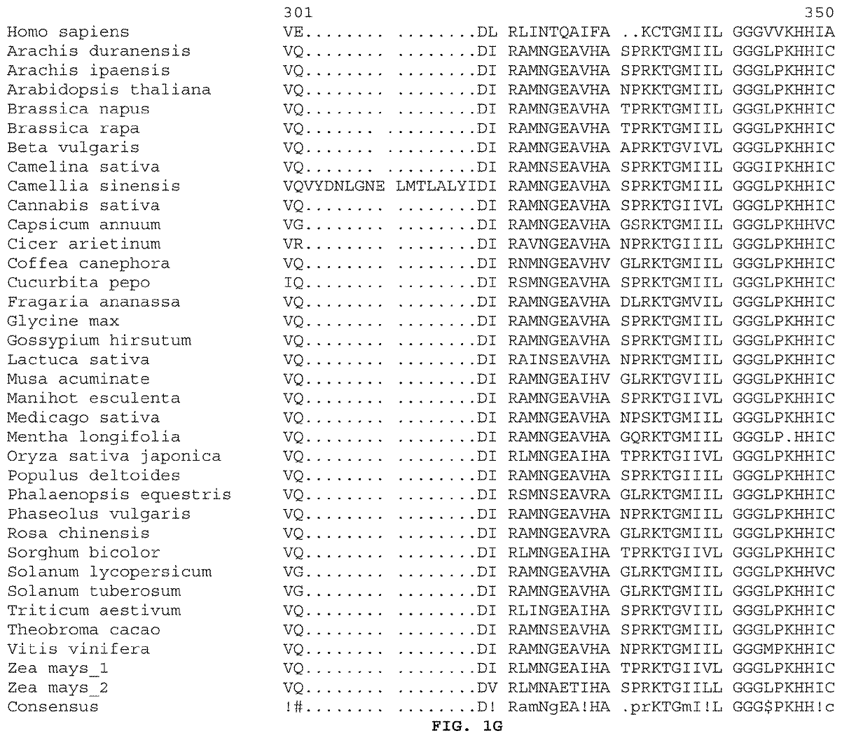

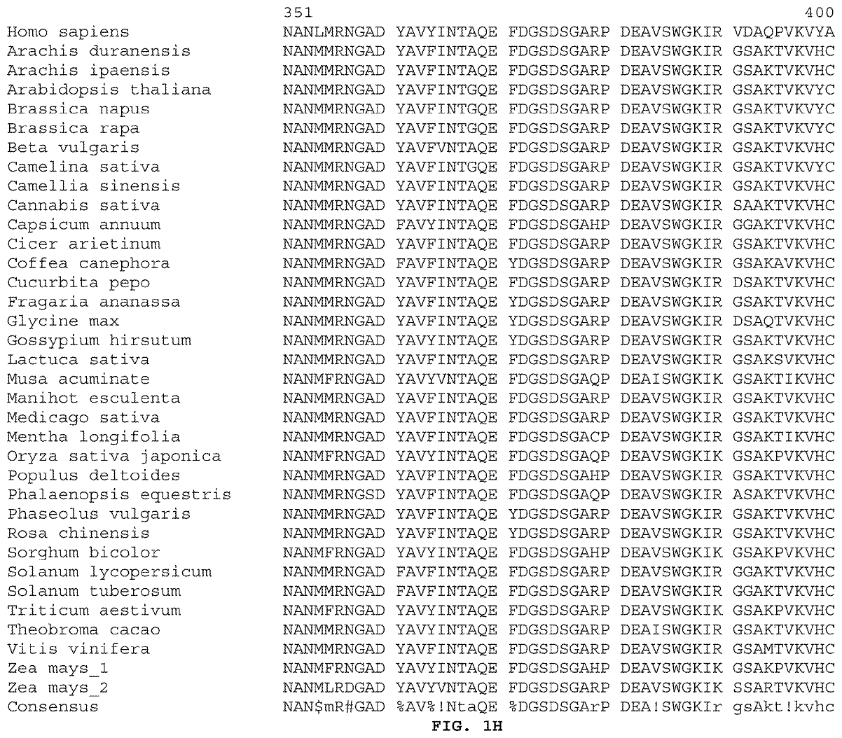

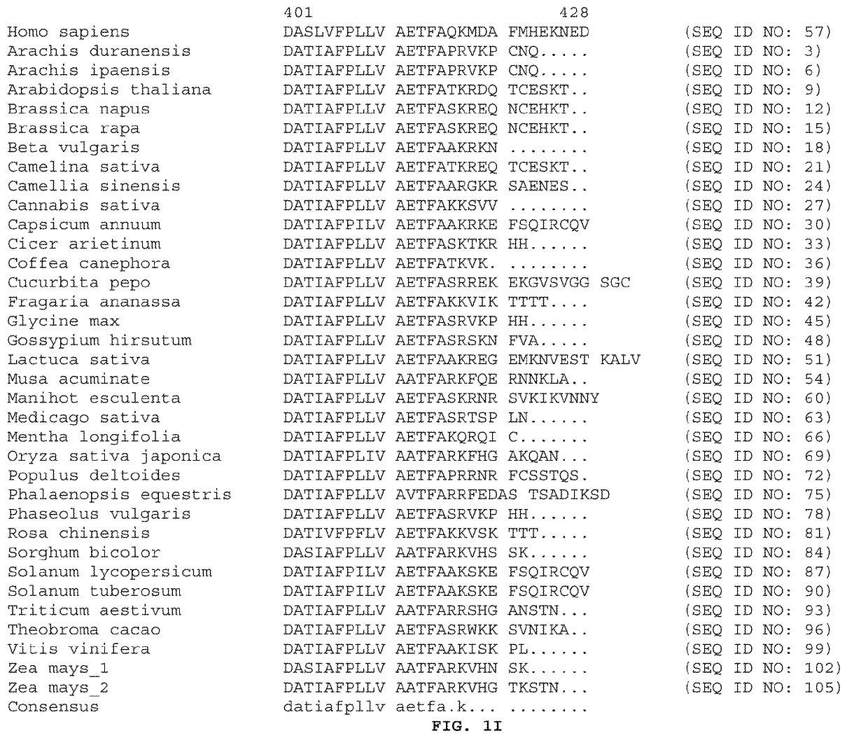

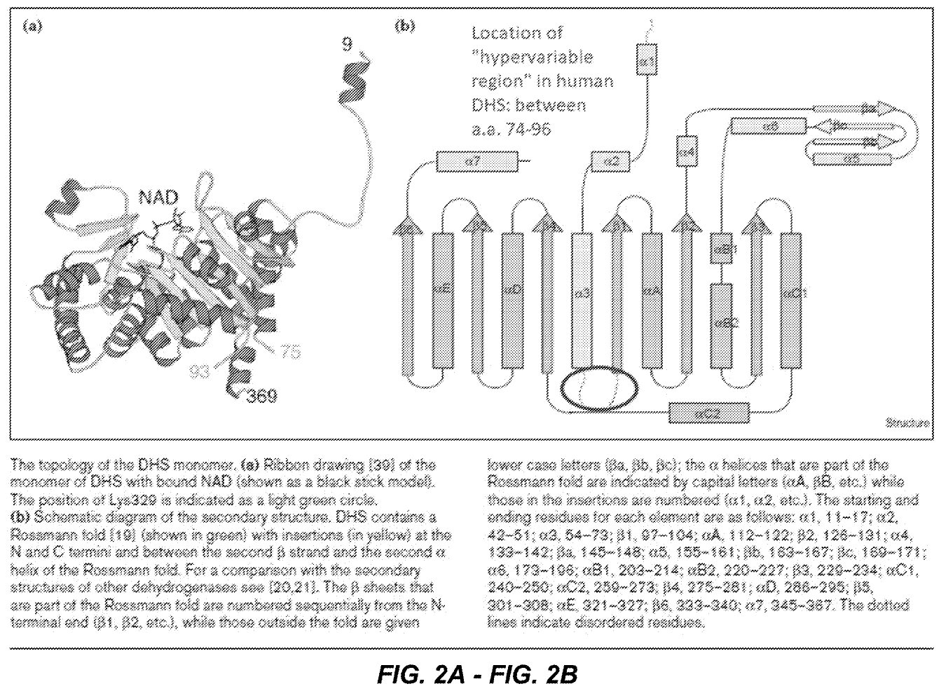

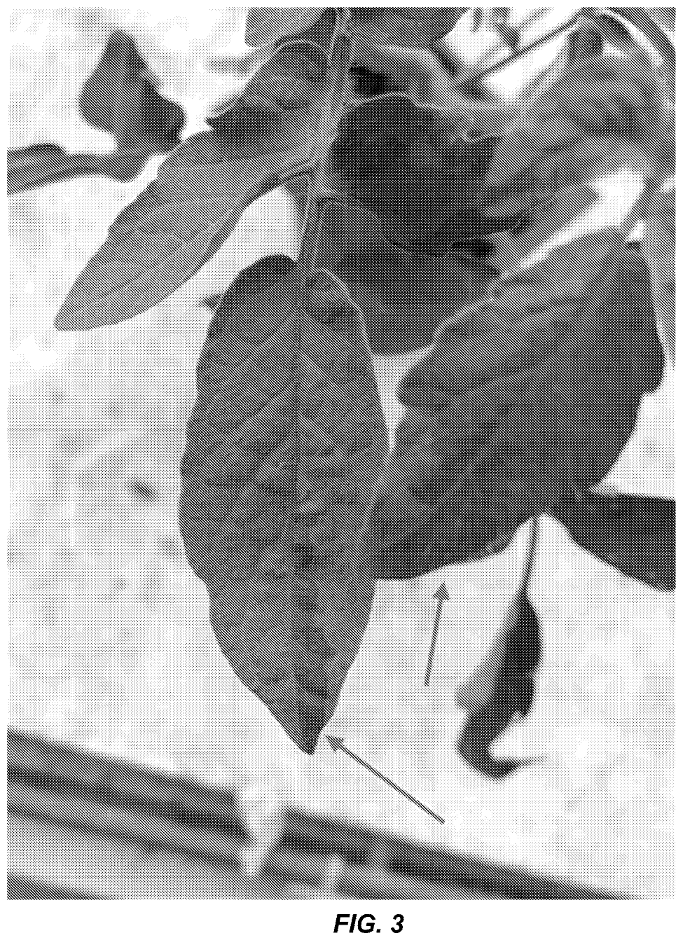

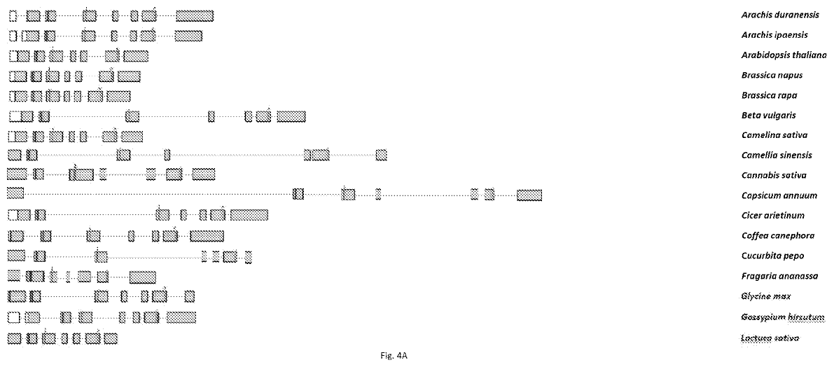

As used herein, a “corresponding residue” refers to any amino acid in a DHS protein, that upon alignment with a second DHS protein amino acid sequence (e.g., human DHS), which is in a different location based on numbering from the N-terminus to C-terminus, and would be in the same location but for the different numbering due to gaps introduced by any sequence alignment. Examples of corresponding residues are described herein and, for example, in FIG. 1. Examples of plant DHS amino acid sequences and corresponding nucleotide sequences include, but are not limited to, the sequences in Table 1.

TABLE 1
Examples of Deoxyhypusine Synthase (DHS) Nucleotide and Amino Acid Sequences

| | GenBank Accession Nos./SEQ ID NO:/References | | |
|---|---|---|---|
| Plant Species | Genomic DNA | cDNA | Protein |
| Arachis duranensis | XP 015966414; | XM_016110928; | XP_015966414; |
| | (SEQ ID NO: 1) | (SEQ ID NO: 2) | (SEQ ID NO: 3) |
| Arachis ipaensis | XP_016203820; | XM_016348334; | XP_016203820; |
| | (SEQ ID NO: 4) | (SEQ ID NO: 5) | (SEQ ID NO: 6) |
| Arabidopsis | NP 196211; | NM_120674; | NP_196211; |
| thaliana | (SEQ ID NO: 7) | (SEQ ID NO: 8) | (SEQ ID NO: 9) |
| Brassica napus | XP_013715226; | XM_013859772; | XP_013715226; |
| | (SEQ ID NO: 10) | (SEQ ID NO: 11) | (SEQ ID NO: 12) |
| Brassica rapa | XP 009122196; | XM_009123948; | XP_009122196; |
| | (SEQ ID NO: 13) | (SEQ ID NO: 14) | (SEQ ID NO: 15) |
| Beta vulgaris | XP 010681287; | XM 010682985; | XP 010681287; |
| subsp. vulgaris | (SEQ ID NO: 16) | (SEQ ID NO: 17) | (SEQ ID NO: 18) |
| Camelina sativa | XP 010452500; | XM_010454198; | XP_010452500; |
| | (SEQ ID NO: 19) | (SEQ ID NO: 20) | (SEQ ID NO: 21) |
| Camellia sinensis | Xia (2017) Molecular | (SEQ ID NO: 23) | Xia (2017) Molecular |
| | Plant 10: 866-877); | | Plant 10: 866-877); |
| | (SEQ ID NO: 22) | | (SEQ ID NO: 24) |
| Cannabis sativa | (SEQ ID NO: 25) | XM_030621917.1; | XP_030477777.1; |
| | | (SEQ ID NO: 26) | (SEQ ID NO: 27) |
| Capsicum annuum | (SEQ ID NO: 28) | (SEQ ID NO: 29) | XP_016560142.1; |
| | | | (SEQ ID NO: 30) |
| Cicer arietinum | XP 004504559.1; | XM_004504502; | XP_004504559; |
| | (SEQ ID NO: 31) | (SEQ ID NO: 32) | (SEQ ID NO: 33) |
| Coffea canephora | (SEQ ID NO: 34) | (SEQ ID NO: 35) | (SEQ ID NO: 36) |
| Cucurbita pepo | (SEQ ID NO: 37) | XM_023679443.1 | XP_023535211.1; |
| | | (SEQ ID NO: 38) | (SEQ ID NO: 39) |
| Fragaria ananassa | Fxa1Cg100564; | (SEQ ID NO: 41) | (SEQ ID NO: 42) |
| | (SEQ ID NO: 40) | | |
| Glycine max | NP 001235604; | NM_001248675; | NP_001235604; |
| | (SEQ ID NO: 43) | (SEQ ID NO: 44) | (SEQ ID NO: 45) |
| Gossypium hirsutum | XP 016700362; | XM_016844873; | XP_016700362; |
| | (SEQ ID NO: 46) | (SEQ ID NO: 47) | (SEQ ID NO: 48) |
| Lactuca sativa | AAU34016; | AY731231; | (SEQ ID NO: 51) |
| | (SEQ ID NO: 49) | (SEQ ID NO: 50) | |
| Musa acuminate | XP 009404132; | XM_009405857; | XP 009404132; |
| | (SEQ ID NO: 52) | (SEQ ID NO: 53) | (SEQ ID NO: 54) |
| Homo sapiens | NP 001921; | (SEQ ID NO: 56) | NP 001921; |
| | (SEQ ID NO: 55) | | (SEQ ID NO: 57) |
| Manihot esculenta | (SEQ ID NO: 58) | XM_021737938.2; | XP_021593630.1; |
| | | (SEQ ID NO: 59) | (SEQ ID NO: 60) |
| Medicago sativa | See, U.S. Pat. No. | See, U.S. Pat. No. | See, U.S. Pat. No. |
| | 8,563,285; | 8,563,285; | 8,563,285; |
| | (SEQ ID NO: 61) | (SEQ ID NO: 62) | (SEQ ID NO: 63) |
| Mentha longifolia | (SEQ ID NO: 64) | (SEQ ID NO: 65) | (SEQ ID NO: 66) |
| Oryza sativa | XP 015628158; | XM_015772672; | XP_015628158; |
| japonica | (SEQ ID NO: 67) | (SEQ ID NO: 68) | (SEQ ID NO: 69) |
| Populus deltoides | See, US | See, US | See, US |
| | 2010/0333233; | 2010/0333233; | 2010/0333233; |
| | (SEQ ID NO: 70) | (SEQ ID NO: 71) | (SEQ ID NO: 72) |
| Phaleaenopsis | XP_020586380.1; | (SEQ ID NO: 74) | (SEQ ID NO: 75) |
| equestris | (SEQ ID NO: 73) | | |
| Phaseolus vulgaris | XP 007152935; | XM_007152873; | XP_007152935; |
| | (SEQ ID NO: 76) | (SEQ ID NO: 77) | (SEQ ID NO: 78) |
| Rosa chinensis | (SEQ ID NO: 79) | XM_024324764.2 | XM_024324764.2 |
| | | (SEQ ID NO: 80) | (SEQ ID NO: 81) |
| Sorghum bicolor | XP 002466487; | XM_002466442; | XP 002466487; |
| | (SEQ ID NO: 82) | (SEQ ID NO: 83) | (SEQ ID NO: 84) |
| Solanum | NP 001234495; | NM_001247566; | NP_001234495; |
| lycopersicum | (SEQ ID NO: 85) | (SEQ ID NO: 86) | (SEQ ID NO: 87) |
| Solanum tuberosum | XP 006348136; | XM_006348074; | XP 006348136; |
| | (SEQ ID NO: 88) | (SEQ ID NO: 89) | (SEQ ID NO: 90) |
| Triticum aestivum | ACP28133; | FJ376389; | ACP28133; |
| | (SEQ ID NO: 91) | (SEQ ID NO: 92) | (SEQ ID NO: 93) |
| Theobroma cacao | XP_007008768.2; | CGD0006914; | XP_007008768.2; |
| | (SEQ ID NO: 94) | (SEQ ID NO: 95) | (SEQ ID NO: 96) |
| Vitis vinifera | (SEQ ID NO: 97) | ENAICBI156001CBI | (SEQ ID NO: 99) |
| | | 15600.3 | |
| | | (SEQ ID NO: 98) | |
| Zea mays 1 | NP 001149084; | NM_001155612; | NP_001149084; |
| | (SEQ ID NO: 100) | (SEQ ID NO: 101) | (SEQ ID NO: 102) |
| Zea mays 2 | NP 001130806; | NM_001137334; | (SEQ ID NO: 105) |
| | (SEQ ID NO: 103) | (SEQ ID NO: 104) | |

For purposes of clarification, the cultivated peanut species (Arachis hypogaea) arose from a hybrid between two wild species of peanut: A. duranensis and A. ipaensis (Seijo et al. (2007) Am. J. Bot. 94 (12)1963-71; Kochert et al. (1996) Am. J. Bot. 83:1282-91; Moretzsohn et al. (2013) Ann. Bot. 111:113-126). The amino acid sequences of the DHS protein between these parental diploids, A. duranensis and A. ipaensis, are identical (SEQ ID NO:3 and SEQ ID NO:6, respectively). Thus, the amino acid sequence of the DHS protein of the cultivated peanut (Arachis hypogaea) is expected to be identical in both parents.

Genome editing endogenous DHS genes by deleting or modifying defined and functionally critical residues, results in plants having no or substantially less DHS protein to activate eIF-5A. As discussed herein, eIF-5A must be activated by DHS to render it biologically useful. Thus, the genome-edited plants will have reduced active eIF-5A by inhibiting or reducing the activity of DHS. The genome-edited plants will have increased biomass, increased seed yield and/or increased seed size, and exhibit greater tolerance to abiotic stress, and, in the case of plants producing perishable fruits or vegetables, extended post-harvest shelf life.

Further evidence to support the contention that DHS and eIF-5A play regulatory roles in senescence was provided by treating carnation flowers with inhibitors that are specific for DHS. Spermidine and eIF-5A are the substrates of DHS reaction ((Park et al. (1993) Biofactors 4:95-104; Park et al. (1997) Biol. Signals. 6:115-123). Several mono-, di-, and polyamines that have structural features similar to spermidine inhibit DHS activity in vitro (Jakus et al. (1993) J. Biol. Chem. 268:13151-13159). Some polyamines, such as spermidine, putrescine, and spermine, have been generally used to extend carnation vase life (Wang and Baker (1980) Hort. Sci. 15:805-806). Flower petal senescence was delayed 6 days after harvest of carnations that were vacuum infiltrated with a transient infection system expressing antisense DHS compared to untreated flowers (Hopkins et al. (2007) New Phytol. 175:201-214).

Post-harvest stress-induced senescence is another cause of agricultural production loss (McCabe et al. (2001) Plant Physiol. 127:505-516). This is true for plants that are partially processed, such as cut lettuce. A symptom of cutting lettuce is browning which is a result of phenolics production (Matile et al. (1999) Annu. Rev. Plant Physiol. Mol. Biol. 50:67-95). A field trial of lettuce with antisense polynucleotides of lettuce eIF-5A (LeIF-5A) or antisense full-length DHS demonstrated that the transgenic lettuce was significantly more resistant to browning after cutting than the control lettuce. It appears that even though stress induced senescence due to harvesting has distinct circuitry, the translational control upstream of browning and likely other senescence symptoms is regulated at least in part by DHS and eIF-5A (Page et al. (2001) Plant Physiol. 125:718-727). Downstream of the regulation of senescence are the execution genes. These are the effectors of senescence and cause the metabolic changes that bring on the senescence syndrome. Downregulating or reducing activity of eIF-5A results in the dampening down of a whole range of symptoms caused by senescence.

### EXAMPLES

### Example 1: Genomic DNA Sequences

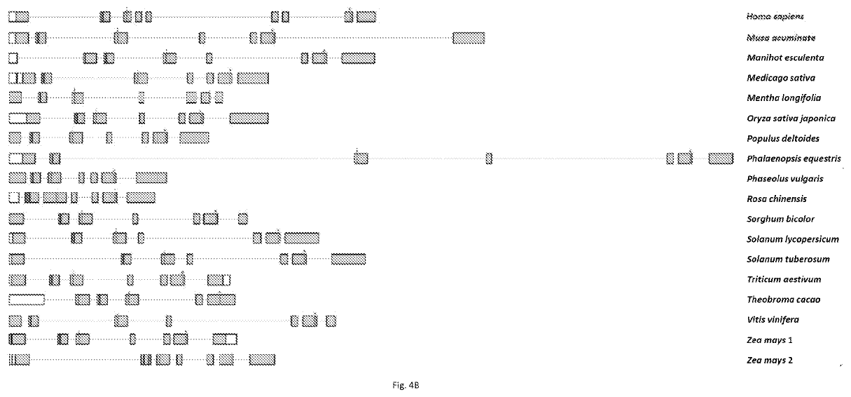

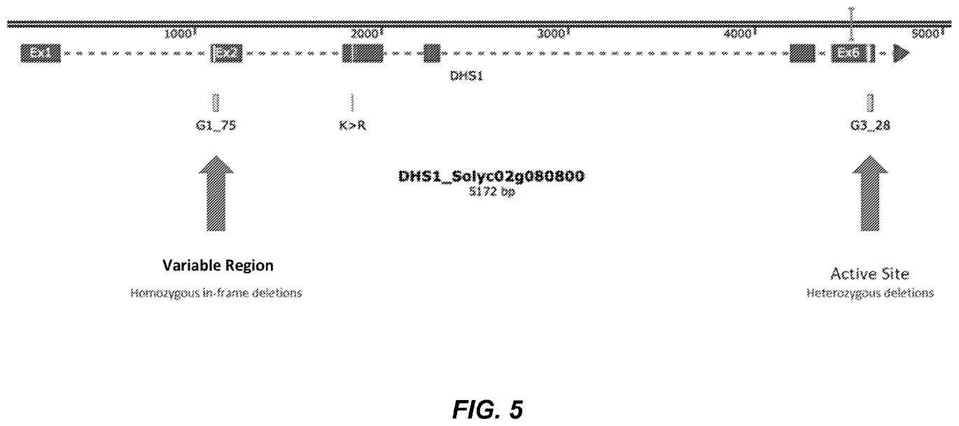

Genomic DNA sequences were identified for 35 DHS genes from 34 plant species and human, and delineated into exons and introns. Two DHS genes or alternate splice variants are shown for Zea mays (maize). Other species (e.g., Solanum lycopersicum) may have more than one DHS gene, even if only one is provided. Guide RNAs for editing DHS genes can be targeted to exons or introns within the genomic DNA. The exon-intron boundaries of 35 of these genomic DNAs are illustrated in FIGS. 4A-4B. The genomic sequences for examples of plant species are provided in Table 1.

### Example 2: ARCUS: an Engineered Homing Endonuclease

An engineered homing endonuclease, known as ARCUS, is engineered to produce a nuclease that would be capable of creating a double-stranded cleavage in a region of S. lycopersicum DHS that is not required for full deoxyhypusine synthase activity. The region specifically targeted for cleavage could include the nucleic acids surrounding the regions that, when translated, includes H76 through E81 (the HELPTE motif; SEQ ID NO:106). The DNA cleavage created by the engineered ARC nuclease would allow the creation of a small in-frame deletion in this targeted region of plant DHS that is known to be hypervariable between plant species. In S. lycopersicum, the activity of the enzyme can be reduced by deleting one or more of the following residues: H76 (no corresponding residue in human DHS—see FIG. 1), E77 (corresponds to E105 in human DHS), L78 (corresponds to P106 in human DHS), P79 (corresponds to L107 in human DHS), T80 (corresponds to 5108 in human DHS), or E81 (corresponds to D110 in human DHS). The DNA cleavage by the ARCUS nuclease allows for the creation of a small deletion in this targeted region of plant DHS that reduces, but does not eliminate, deoxyhypusine synthase (DHS) activity. Amino acid sequences are highly conserved on both sides of this hypervariable 6-amino acid region.

### Example 3: Engineered Transcription Activator-Like Effector Nucleases (TALENs)

An engineered transcription activator-like effector (TALE) combined with a functional domain, e.g., a FokI nuclease (TALEN), is engineered to produce a nuclease that would be capable of creating a double-stranded cleavage in a region of S. lycopersicum DHS that is not required for full deoxyhypusine synthase activity. Similarly, a pair of TALENs fused to Clo51, a nuclease that only functions when the distance between DNA binding sites is appropriate, is designed flanking a target site. The region specifically targeted for cleavage could include the nucleic acids surrounding the regions that, when translated, includes H76 through E81 (the HELPTE motif; SEQ ID NO: 106). The DNA cleavage created by the engineered TALE would allow the creation of a small in-frame deletion in this targeted region of plant DHS that is known to be hypervariable between plant species. In S. lycopersicum, the activity of the enzyme can be reduced by deleting one or more of the following residues: H76 (no corresponding residue in human DHS—see FIG. 1), E77 (corresponds to E105 in human DHS), L78 (corresponds to P106 in human DHS), P79 (corresponds to L107 in human DHS), T80 (corresponds to S108 in human DHS), or E77 (corresponds to D110 in human DHS). The DNA cleavage by the engineered TALEN allows for the creation of a small deletion in this targeted region of plant DHS that reduces, but doesn't eliminate, deoxyhypusine synthase activity. Amino acid sequences are highly conserved on both sides of this hypervariable 6-amino acid region.

### Example 4: sgRNAs Capable of Double-Strand Cleavage

A sgRNA is engineered to produce a guide RNA capable of creating a double-stranded cleavage, when introduced into the plant along with Cas9 (CRISPR-Cas9 system) or another nuclease, in a region of S. lycopersicum DHS that is not required for full deoxyhypusine synthase activity. The region specifically targeted for cleavage could include the nucleic acids surrounding the regions that, when translated, includes H76 through E81 (the HELPTE motif; SEQ ID NO: 106). The DNA cleavage created by the sgRNA and CRISPR nuclease would allow the creation of a small in-frame deletion in this targeted region of plant DHS that is known to be hypervariable between plant species. In S. lycopersicum, the activity of the enzyme can be reduced by deleting one or more of the following residues: H76 (no corresponding residue in human DHS—see FIG. 1c), E77 (corresponds to E105 in human DHS), L78 (corresponds to P106 in human DHS), P79 (corresponds to L107 in human DHS), T80 (corresponds to S108 in human DHS), or E81 (corresponds to D1110 in human DHS). The nucleic acid sequence for the 6-amino acid hypervariable region within the Solanum lycopersicum DHS1 gene sequence is CATGAGCTGCCCACGGAG (SEQ ID NO:107). The DNA cleavage by the sgRNA and CRISPR nuclease allows for the creation of a small deletion in this targeted region of plant DHS that reduces, but doesn't eliminate, deoxyhypusine synthase activity. Amino acid sequences are highly conserved on both sides of this hypervariable 6-amino acid region.

### Example 5: Editing Pre-Determined Genomic Loci in Solanum lycopersicum

One or more gRNAs is designed to anneal with a desired site in the tomato genome and to allow for interaction with one or more Cas9 or other CRISPR double stranded nuclease proteins. These gRNAs are cloned in a vector such that they are operably linked to a promoter that is operable in a plant cell (the gRNA cassette). One or more genes encoding a Cas9 or other CRISPR double stranded nuclease protein is cloned in a vector such that they are operably linked to a promoter that is operable in a plant cell (the CRISPR nuclease cassette). The gRNA cassette and the CRISPR nuclease cassette are each cloned into a vector that is suitable for plant transformation, and this vector is subsequently transformed into Agrobacterium cells. These cells are brought into contact with tomato tissue that is suitable for transformation. Following this incubation with the Agrobacterium cells, the tomato cells are cultured on a tissue culture medium that is suitable for regeneration of intact plants with selection against Agrobacterium cells. Tomato plants are regenerated from the cells that were brought into contact with Agrobacterium cells harboring the vector that contained the CRISPR nuclease cassette and gRNA cassette. Following regeneration of the tomato plants, plant tissue is harvested and DNA is extracted from the tissue. DNA sequencing assays are performed, as appropriate, to determine whether a change in the DNA sequence has occurred at the desired genomic location.

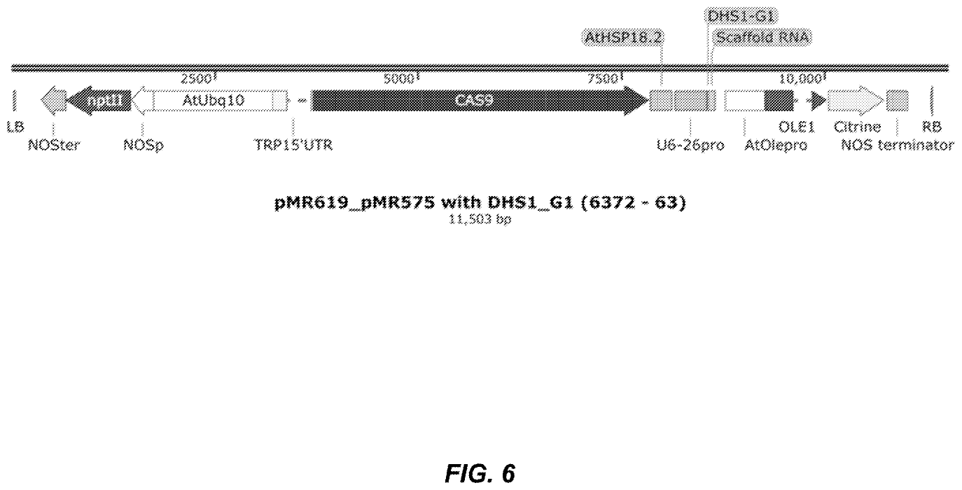

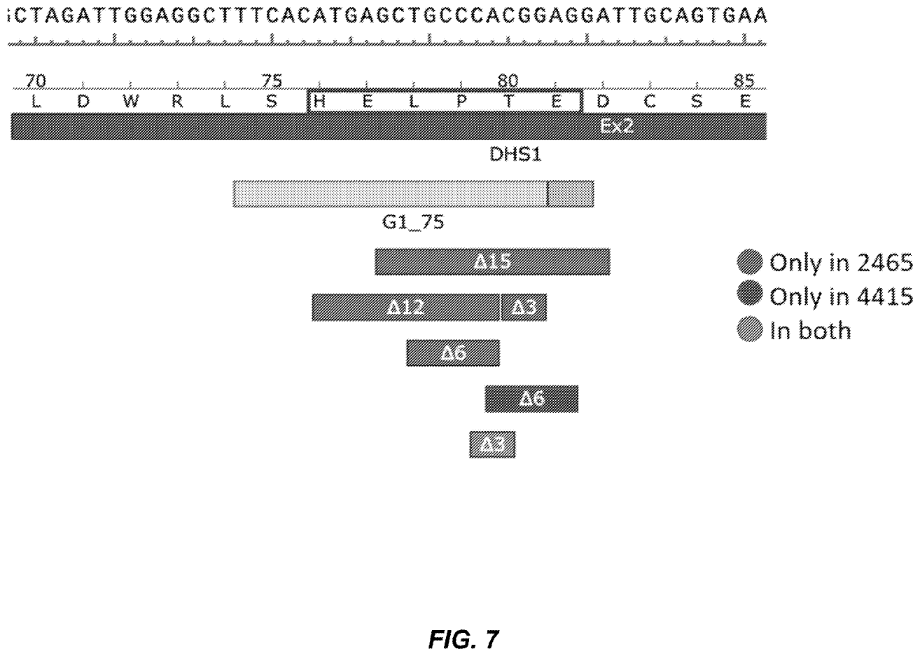

Plasmid construction. Two gRNAs were designed for the DHS1 gene of Solanum lycopersicum using CRISPOR, which is a program that helps design, evaluate and clone guide sequences for the CRISPR/Cas9 system. The targets chosen were 20 bp at position 1027-1046 (TCACATGAGCTGCCCACGG—referred to as DHS1_G1 (SEQ ID NO:108)) and at position 4527-4546 (GTATCATGGGGAAAGATACG—referred to as DHS1_G3 (SEQ ID NO:109)). Cloning into pEn_Chimera was performed [Fauser et al. (2014) The Plant Cell 29: 843-853]. Both CRISPR/Cas9-based nucleases and nickases can be used efficiently for genome engineering in Arabidopsis thaliana. Briefly, two 23 bp overlapping oligos were designed for each gRNA, in which the first 2 bp of the target were changed to GG (see, Table 2). These oligos were annealed and cloned into Bpil-digested pEn_Chimera. The resulting plasmids were used in an LR recombination (Thermo Fisher Scientific, Waltham, MA, USA) to transfer the AtU6-26p-DHS1_G1_sgRNA or AtU6-26p-DHS1_G3_sgRNA cassette into pMR575 binary vector. The vector pMR575 contains AtUBQ10p_TRP15′UTR (Cor15a11L) (Gallegos & Rose (2017) The Plant Cell 29: 843-853)]. Intron DNA sequences can be more important than the proximal promoter in determining the site of transcript initiation (Fauser et al. (2014) The Plant Cell 29:843-853) driving the SpCas9, AtOLEp-AtOLE1-Citrine as a visible marker (expressed in mature embryos), and a kanamycin resistance marker. These final vectors are termed pMR618 (carrying G3) and pMR619 (carrying G1—see FIG. 6). To generate edited tomato plants, pMR618 and pMR619 were introduced into Agrobacterium tumefaciens strain EHA105 and were used for stable transformation of tomato cotyledons of the varieties TF2465 and TF4415, employing the kanamycin marker for selection, essentially using the method described (Bari et al. (2019) Scientific Reports 9:11438).

TABLE 2
Oligonucleotides used to build construct RNA

| | | |
|---|---|---|
| DHS1_G1.For | attggCACATGAGCTGCCCACGG | SEQ ID |
| | | NO: 110 |
| DHS1_G1.Rev | aaacCCGTGGGCAGCTCATGTGC | SEQ ID |
| | | NO: 111 |
| DHS1_G3.For | attggATCATGGGGAAAGATAC | SEQ ID |
| | | NO: 112 |
| DHS1_G3.Rev | aaacCGTATCTTTCCCCATGATC | SEQ ID |
| | | NO: 113 |

Genotyping. Transgenic T0 plants and their progeny were genotyped for mutations in DHL1 by Sanger sequencing of the PCR-amplified target regions using the primers listed below. The Sanger sequence chromatograms were decomposed by web tools from TIDE or ICE.

TABLE 3
Genotyping Primers

| | | |
|---|---|---|
| G1_Screen.R1 | AGCATAGCAGCGATTCAGTGC | SEQ ID NO: 114 |
| G3_Screen.F1 | GGATGGATCACTTGGTGACATGC | SEQ ID NO: 115 |
| G3_Screen.R1 | CATCTAAGCTCTTCACTTTCGAAGAGG | SEQ ID NO: 116 |

The presence or absence of the transgene was confirmed via PCR using the following primers, assaying presence or absence of the predicted PCR band.

TABLE 4
Primers to Confirm Presence of Absence of Transgene

| | | |
|---|---|---|
| NptII_Rev2 | AACGTCGAGCACAGCTGCGC | SEQ ID |
| | | NO: 117 |
| AtUBQ10p_ | TGAAGAAGGGAACTTATCCGGTCC | SEQ ID |
| Screen.Rev | | NO: 118 |
| Citrine.For3 | GACAACCACTACCTGAGCTACCAG | SEQ ID |
| | | NO: 119 |
| QB0049 | CCCAGTCACGACGTTGTAAAACG | SEQ ID |
| | | NO: 120 |
| NptII_For2 | CGATGCCTGCTTGCCGAATATCATGG | SEQ ID |
| | | NO: 121 |
| pDe_Cas9_ | GTGAGCGGATAACAATTTCACACAGG | SEQ ID |
| LB.FOR | | NO: 122 |

Plant propagation and phenotyping. Plants were growing in a greenhouse at 15C-26C with natural day light. In winter, the plants received supplemental light from 5 am-10 am and 5 pm-10 pm. No obvious developmental phenotypes were observed pre-fruiting.

T₁ or T₂ plants that are homozygous or biallelic (=two mutant alleles) were used for comparison to their wild type progenitor. A series of 4 small, in-frame, biallelic deletion mutations in the hypervariable region of the DHS1 gene were isolated in two different tomato germplasm sources: TF2465 (plum) and TF4415 (round). These mutations are described in Table 5.

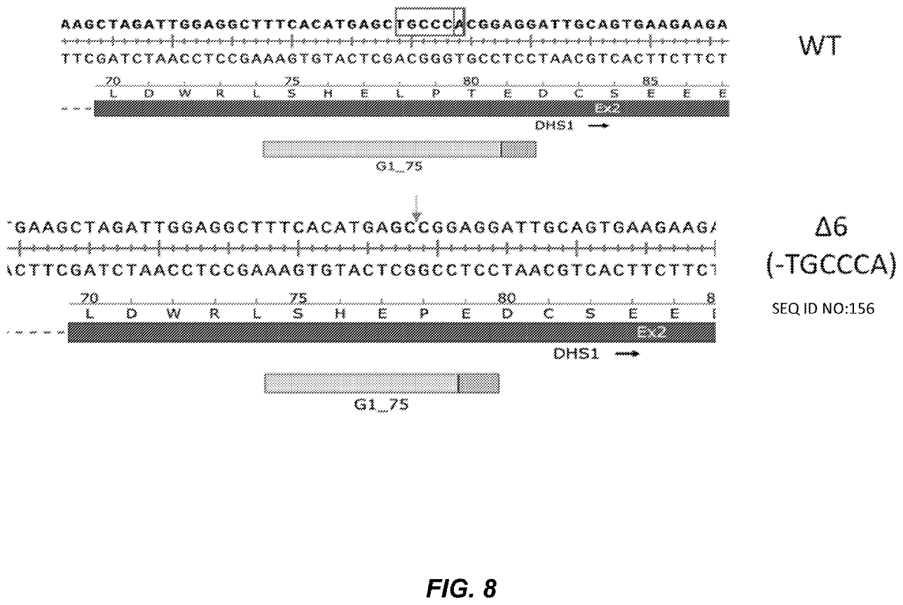

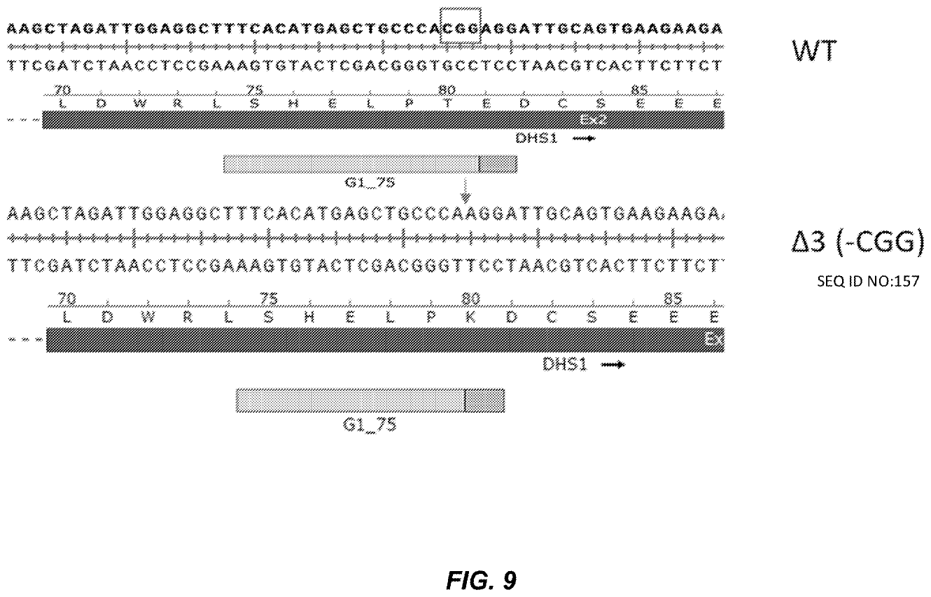

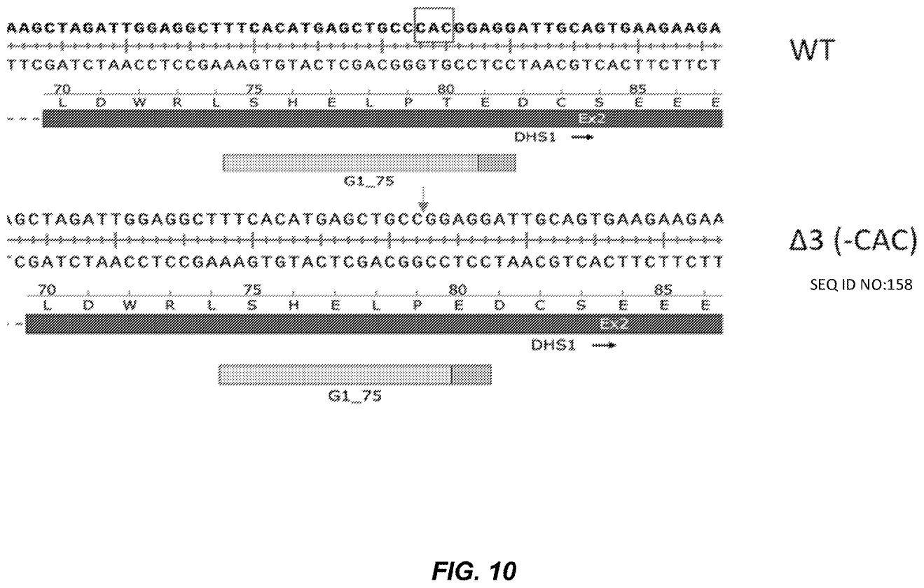

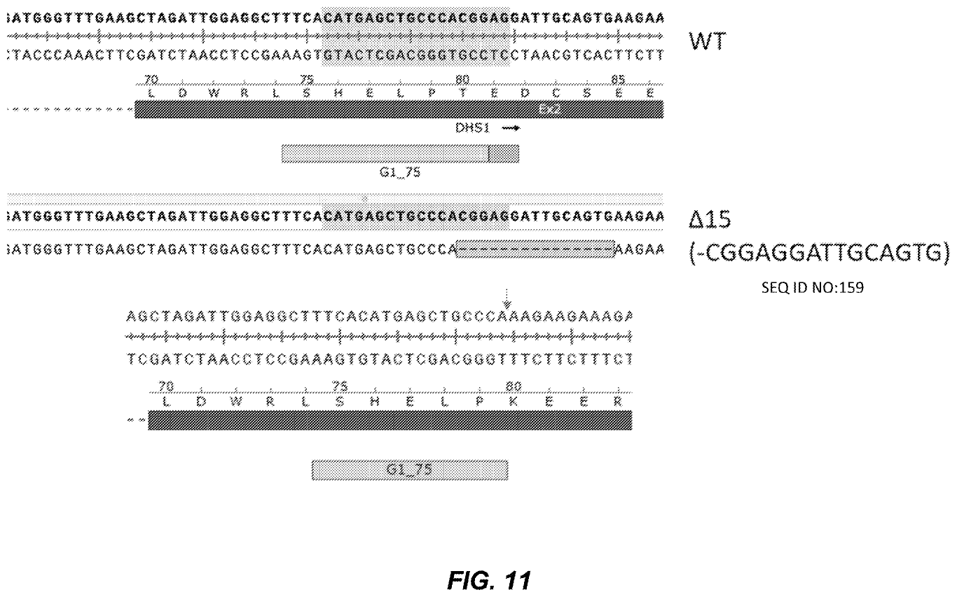

TABLE 5
Biallelic DHS1 deletion mutations from TF2465 (plum) and TF4415 (round)

| | | T2 DHS1 | | |
|---|---|---|---|---|
| Line | Background | genotype | In-Frame Mutation | |
| DHS1 4-27 | TF2465 | -6/-6 | -TGCCCA | FIG. 8 |
| DHS1 73-10 | TF2465 | -3/-3 | -CGG | FIG. 9 |
| DHS1 135-3 | TF4415 | -3/-3 | -CAC | FIG. 10 |
| DHS1 158-3 | TF4415 | -15/-15 | -CGGAGGATTGCAGTG | FIG. 11 |
| | | | (SEQ ID NO: 156) | |

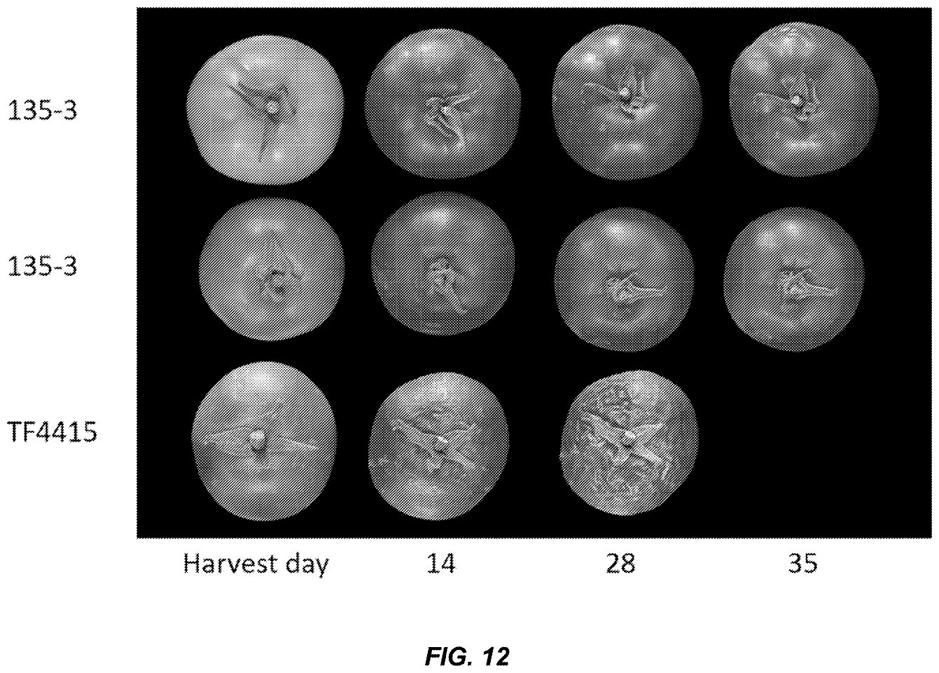

The tomatoes of DHS1_135-3 were harvested at different stages (green/breaker/turning/pink/red) and kept in a tray on the bench in the lab at room temperature. Photos of the tomatoes were taken weekly. Wild type and mutant tomatoes of the same variety, harvested at the same stage of fruit development, were compared over time (see FIG. 12). It is apparent that this small 3 base pair homozygous deletion mutation, that removed a single amino acid in the hypervariable region, reduced activity of the DHS1 gene in tomato sufficient to extend the shelf life of this mutant fruit by 4- to 5-fold longer than the wild type control, with no other obvious phenotypic effects on the plant. Similar results were observed with the other 3 mutations.

### Example 6: Particle Bombardment to Introduce Cassettes

Alternatively, particle bombardment is used to introduce the CRISPR nuclease cassette and gRNA cassette into tomato cells. Vectors containing a CRISPR nuclease cassette and a gRNA cassette are coated onto gold beads or titanium beads that are then used to bombard tomato tissue that is suitable for regeneration. Following bombardment, the tomato tissue is transferred to tissue culture medium for regeneration of tomato plants. Following regeneration of the tomato plants, plant tissue is harvested and DNA is extracted from the tissue. T7EI assays and/or sequencing assays are performed, as appropriate, to determine whether a change in the DNA sequence has occurred at the desired genomic location.

### Example 7: Deleting DNA from a Pre-Determined Genomic Locus Using Non-Homologous End Joining

A first gRNA is designed to anneal with a first desired site in the genome of a plant of interest and to allow for interaction with one or more Cas9 or other CRISPR double stranded nuclease proteins. A second gRNA is designed to anneal with a second desired site in the genome of a plant of interest and to allow for interaction with one or more CRISPR nuclease proteins. Each of these gRNAs is operably linked to a promoter that is operable in a plant cell and is subsequently cloned into a vector that is suitable for plant transformation. One or more genes encoding a Cas9 or other CRISPR double stranded nuclease protein is cloned in a vector such that they are operably linked to a promoter that is operable in a plant cell (the “CRISPR nuclease cassette”). The CRISPR nuclease cassette and the gRNA cassettes are cloned into a single plant transformation vector that is subsequently transformed into Agrobacterium cells. These cells are brought into contact with plant tissue that is suitable for transformation. Following this incubation with the Agrobacterium cells, the plant cells are cultured on a tissue culture medium that is suitable for regeneration of intact plants with selection against Agrobacterium cells. Alternatively, the vector containing the CRISPR nuclease cassette and the gRNA cassettes is coated onto gold or titanium beads suitable for bombardment of plant cells. The cells are bombarded and are then transferred to tissue culture medium that is suitable for the regeneration of intact plants. The gRNA-CRISPR nuclease complexes effect double-stranded breaks at the desired genomic loci and in some cases the DNA repair machinery causes the DNA to be repaired in such a way that some native DNA sequence that was located near or within the gRNA sequence is deleted. Plants are regenerated from the cells that are brought into contact with Agrobacterium cells harboring the vector that contains the CRISPR nuclease cassette and gRNA cassettes or are bombarded with beads coated with this vector. Following regeneration of the plants, plant tissue is harvested and DNA is extracted from the tissue. Sequencing assays are performed, as appropriate, to determine whether DNA has been deleted from the desired genomic location or locations.

### Example 8: Genome Editing of the DHS1 Loci in Soybeans (Glycine max)

Guide RNAs (gRNAs) will be designed to anneal with a chosen site in the soybean genome and allowed to interact with one or more Cas9 or other CRISPR double-stranded nuclease proteins. These gRNAs will be cloned in a vector and operably-linked to a promoter, e.g., the gRNA cassette, in a plant cell. One or more genes encoding a Cas9 or other CRISPR double-stranded nuclease protein will be cloned in a vector and operably linked to a promoter, e.g., the CRISPR nuclease cassette, in a plant cell (the CRISPR nuclease cassette). The gRNA and CRISPR nuclease cassettes will be cloned into a vector that is suitable for plant transformation, and this vector will be subsequently transformed into Agrobacterium cells. These cells will be brought into contact with soybean tissue that is suitable for transformation. Following incubation with Agrobacterium cells, the soybean cells will be cultured on a tissue culture medium that is suitable for regeneration of intact plants with selection against Agrobacterium cells. Soybean plants will be regenerated from the cells that were brought into contact with Agrobacterium cells harboring the vector that contained the gRNA and CRISPR nuclease cassettes. Following regeneration of the soybean plants, DNA will be extracted from harvested plant tissue. DNA sequencing assays will be performed to determine whether a change has occurred in the DNA sequence at the targeted genomic location.

Plasmid construction, genotyping, and plant propagation. As described in Example 5, at least one gRNA will be designed to be internal or proximal to the soybean genomic sequence encoding the corresponding 6-amino acid hypervariable region of a DHS1 gene in this species: DEPVAE (SEQ ID NO:123). The first step will be to sequence the relevant region of the DHS1 gene in the actual soybean cultivar used for genome editing to confirm the absence of single nucleotide polymorphisms (SNPs) which would affect the annealing efficiency of a gRNA. The nucleic acid sequence for the 6-amino acid hypervariable region within the Glycine max DHS1 gene sequence is GATGAACCCGTAGCTGAG (SEQ ID NO:124) and will be used, along with flanking genomic sequence, to generate candidate gRNA sequences. An example of one alternative nucleic acid sequence to generate gRNA is the non-coding strand gRNA sequence (PAM sequence underlined) GGGCATCGACTCCTAACGTCAC (SEQ ID NO:125).

Stable transformation of soybean tissue will be performed with the appropriate selectable and/or scorable markers after plasmid construction, sequence confirmation, and transfer into Agrobacterium vector and host. Healthy callus tissues will be shooted and rooted using the appropriate hormone-enhanced solid media, and T0 soybean plants will be regenerated and their progeny genotyped for the targeted mutations in the DHS1 gene by Next Generation Sequencing (NGS) or Sanger sequencing of PCR-amplified target regions using flanking primers.

In one embodiment, the T1 plants with confirmed soybean DHS1 sequence deletions will be selfed, and the T2 plants will be screened for the presence of homozygous DHS1 mutations and absence of transgenes, e.g., null segregants. The selected T2 plants will be selfed and T3 seeds will be propagated to test for increased shelf life, higher yields, and/or greater abiotic/biotic stress tolerance compared to wild-type controls.

### Example 9: Genome Editing of the DHS1 Loci in Common Bean (Phaseolus vulgaris)

Guide RNAs (gRNAs) will be designed to anneal with a chosen site in the common bean genome and allowed to interact with one or more Cas9 or other CRISPR double-stranded nuclease proteins. These gRNAs will be cloned in a vector and operably-linked to a promoter, e.g., the gRNA cassette, in a plant cell. One or more genes encoding a Cas9 or other CRISPR double-stranded nuclease protein will be cloned in a vector and operably linked to a promoter, e.g., the CRISPR nuclease cassette, in a plant cell (the CRISPR nuclease cassette). The gRNA and CRISPR nuclease cassettes will be cloned into a vector that is suitable for plant transformation, and this vector will be subsequently transformed into Agrobacterium cells. These cells will be brought into contact with common bean tissue that is suitable for transformation. Following incubation with Agrobacterium cells, the common bean cells will be cultured on a tissue culture medium that is suitable for regeneration of intact plants with selection against Agrobacterium cells. Common bean plants will be regenerated from the cells that were brought into contact with Agrobacterium cells harboring the vector that contained the gRNA and CRISPR nuclease cassettes. Following regeneration of the common bean plants, DNA will be extracted from harvested plant tissue. DNA sequencing assays will be performed to determine whether a change has occurred in the DNA sequence at the targeted genomic location.

Plasmid construction, genotyping, and plant propagation. As described in Example 5, at least one gRNA will be designed to be internal or proximal to the common bean genomic sequence encoding the corresponding 6-amino acid hypervariable region of a DHS1 gene in this species: DEAVTE (SEQ ID NO:126).

The first step will be to sequence the relevant region of the DHS1 gene in the actual common bean cultivar used for genome editing to confirm the absence of single nucleotide polymorphisms (SNPs) which would affect the annealing efficiency of a gRNA. The nucleic acid sequence for the 6-amino acid hypervariable region within the Phaseolus vulgaris DHS1 gene sequence is GATGAAGCCGTGACTGAG (SEQ ID NO:127) and will be used, along with flanking genomic sequence, to generate candidate gRNA sequences. An example of one alternative nucleic acid sequence to generate gRNA is the non-coding strand gRNA sequence (PAM sequence underlined) GGCACTGACTCCTAACGTCACTG (SEQ ID NO:128). Another example of an alternative nucleic acid sequence to generate gRNA is the coding strand gRNA sequence (PAM sequence underlined) AGTTGATGAAGCCGTGACTGAGG (SEQ ID NO:129).

Stable transformation of common bean tissue will be performed with the appropriate selectable and/or scorable markers after plasmid construction, sequence confirmation, and transfer into Agrobacterium vector and host. Healthy callus tissues will be shooted and rooted using the appropriate hormone-enhanced solid media, and T0 common bean plants will be regenerated and their progeny genotyped for the targeted mutations in the DHS1 gene by Next Generation Sequencing (NGS) or Sanger sequencing of PCR-amplified target regions using flanking primers.

In one embodiment, the T1 plants with confirmed common bean DHS1 sequence deletions will be selfed, and the T2 plants will be screened for the presence of homozygous DHS1 mutations and absence of transgenes, e.g., null segregants. The selected T2 plants will be selfed and T3 seeds will be propagated to test for increased shelf life, higher yields, and/or greater abiotic/biotic stress tolerance compared to wild-type controls.

### Example 10: Genome Editing of the DHS1 Loci in Strawberry (Fragaria ananassa)

Guide RNAs (gRNAs) will be designed to anneal with a chosen site in the strawberry genome and allowed to interact with one or more Cas9 or other CRISPR double-stranded nuclease proteins. These gRNAs will be cloned in a vector and operably-linked to a promoter, e.g., the gRNA cassette, in a plant cell. One or more genes encoding a Cas9 or other CRISPR double-stranded nuclease protein will be cloned in a vector and operably linked to a promoter, e.g., the CRISPR nuclease cassette, in a plant cell (the CRISPR nuclease cassette). The gRNA and CRISPR nuclease cassettes will be cloned into a vector that is suitable for plant transformation, and this vector will be subsequently transformed into Agrobacterium cells. These cells will be brought into contact with strawberry tissue that is suitable for transformation. Following incubation with Agrobacterium cells, the strawberry cells will be cultured on a tissue culture medium that is suitable for regeneration of intact plants with selection against Agrobacterium cells. Strawberry plants will be regenerated from the cells that were brought into contact with Agrobacterium cells harboring the vector that contained the gRNA and CRISPR nuclease cassettes. Following regeneration of the strawberry plants, DNA will be extracted from harvested plant tissue. DNA sequencing assays will be performed to determine whether a change has occurred in the DNA sequence at the targeted genomic location.

Plasmid construction, genotyping, and plant propagation. As described in Example 5, at least one gRNA will be designed to be internal or proximal to the strawberry genomic sequence encoding the corresponding 6-amino acid hypervariable region of a DHS1 gene in this species: DEAVAD (SEQ ID NO:130).

The first step will be to sequence the relevant region of the DHS1 gene in the actual strawberry cultivar used for genome editing to confirm the absence of single nucleotide polymorphisms (SNPs) which would affect the annealing efficiency of a gRNA. The nucleic acid sequence for the 6-amino acid hypervariable region within the Fragaria ananassa DHS1 gene sequence is GATGAGGCTGTAGCTGAC (SEQ ID NO:131) and will be used, along with flanking genomic sequence, to generate candidate gRNA sequences. An example of one alternative nucleic acid sequence to generate gRNA is the coding strand gRNA sequence (PAM sequence underlined) GAGGCTGTAGCTGACGACTGCGG (SEQ ID NO:132).

Stable transformation of strawberry tissue will be performed with the appropriate selectable and/or scorable markers after plasmid construction, sequence confirmation, and transfer into Agrobacterium vector and host. Healthy callus tissues will be shooted and rooted using the appropriate hormone-enhanced solid media, and T0 strawberry plants will be regenerated and their progeny genotyped for the targeted mutations in the DHS1 gene by Next Generation Sequencing (NGS) or Sanger sequencing of PCR-amplified target regions using flanking primers.

In one embodiment, the T1 plants with confirmed strawberry DHS1 sequence deletions will be selfed, and the T2 plants will be screened for the presence of homozygous DHS1 mutations and absence of transgenes, e.g., null segregants. The selected T2 plants will be selfed and T3 seeds will be propagated to test for increased shelf life, higher yields, and/or greater abiotic/biotic stress tolerance compared to wild-type controls.

### Example 11: Genome Editing of the DHS1 Loci in Pepper (Capsicum annuum)

Guide RNAs (gRNAs) will be designed to anneal with a chosen site in the pepper genome and allowed to interact with one or more Cas9 or other CRISPR double-stranded nuclease proteins. These gRNAs will be cloned in a vector and operably-linked to a promoter, e.g., the gRNA cassette, in a plant cell. One or more genes encoding a Cas9 or other CRISPR double-stranded nuclease protein will be cloned in a vector and operably linked to a promoter, e.g., the CRISPR nuclease cassette, in a plant cell (the CRISPR nuclease cassette). The gRNA and CRISPR nuclease cassettes will be cloned into a vector that is suitable for plant transformation, and this vector will be subsequently transformed into Agrobacterium cells. These cells will be brought into contact with pepper tissue that is suitable for transformation. Following incubation with Agrobacterium cells, the pepper cells will be cultured on a tissue culture medium that is suitable for regeneration of intact plants with selection against Agrobacterium cells. Pepper plants will be regenerated from the cells that were brought into contact with Agrobacterium cells harboring the vector that contained the gRNA and CRISPR nuclease cassettes. Following regeneration of the pepper plants, DNA will be extracted from harvested plant tissue. DNA sequencing assays will be performed to determine whether a change has occurred in the DNA sequence at the targeted genomic location.

Plasmid construction, genotyping, and plant propagation. As described in Example 5, at least one gRNA will be designed to be internal or proximal to the pepper genomic sequence encoding the corresponding 6-amino acid hypervariable region of a DHS1 gene in this species: HEVPTE (SEQ ID NO:133).

The first step will be to sequence the relevant region of the DHS1 gene in the actual pepper cultivar used for genome editing to confirm the absence of single nucleotide polymorphisms (SNPs) which would affect the annealing efficiency of a gRNA. The nucleic acid sequence for the 6-amino acid hypervariable region within the Capsicum annuum DHS1 gene sequence is CATGAGGTTCCTACTGAG (SEQ ID NO:134) and will be used, along with flanking genomic sequence, to generate candidate gRNA sequences. An example of one alternative nucleic acid sequence to generate gRNA is the coding strand gRNA sequence (PAM sequence underlined) TTCACATGAGGTTCCTACTGAGG (SEQ ID NO:135). Another example of an alternative nucleic acid sequence to generate gRNA is the non-coding strand gRNA sequence (PAM sequence underlined) GGATGACTCCTAACGTCACTTCT (SEQ ID NO:136).

Stable transformation of pepper tissue will be performed with the appropriate selectable and/or scorable markers after plasmid construction, sequence confirmation, and transfer into Agrobacterium vector and host. Healthy callus tissues will be shooted and rooted using the appropriate hormone-enhanced solid media, and T0 pepper plants will be regenerated and their progeny genotyped for the targeted mutations in the DHS1 gene by Next Generation Sequencing (NGS) or Sanger sequencing of PCR-amplified target regions using flanking primers.

In one embodiment, the T1 plants with confirmed pepper DHS1 sequence deletions will be selfed, and the T2 plants will be screened for the presence of homozygous DHS1 mutations and absence of transgenes, e.g., null segregants. The selected T2 plants will be selfed and T3 seeds will be propagated to test for increased shelf life, higher yields, and/or greater abiotic/biotic stress tolerance compared to wild-type controls.

### Example 12: Genome Editing of the DHS1 Loci in Zucchini (Cucurbita pepo)

Guide RNAs (gRNAs) will be designed to anneal with a chosen site in the zucchini genome and allowed to interact with one or more Cas9 or other CRISPR double-stranded nuclease proteins. These gRNAs will be cloned in a vector and operably-linked to a promoter, e.g., the gRNA cassette, in a plant cell. One or more genes encoding a Cas9 or other CRISPR double-stranded nuclease protein will be cloned in a vector and operably linked to a promoter, e.g., the CRISPR nuclease cassette, in a plant cell (the CRISPR nuclease cassette). The gRNA and CRISPR nuclease cassettes will be cloned into a vector that is suitable for plant transformation, and this vector will be subsequently transformed into Agrobacterium cells. These cells will be brought into contact with zucchini tissue that is suitable for transformation. Following incubation with Agrobacterium cells, the zucchini cells will be cultured on a tissue culture medium that is suitable for regeneration of intact plants with selection against Agrobacterium cells. Zucchini plants will be regenerated from the cells that were brought into contact with Agrobacterium cells harboring the vector that contained the gRNA and CRISPR nuclease cassettes. Following regeneration of the zucchini plants, DNA will be extracted from harvested plant tissue. DNA sequencing assays will be performed to determine whether a change has occurred in the DNA sequence at the targeted genomic location.

Plasmid construction, genotyping, and plant propagation. As described in Example 5, at least one gRNA will be designed to be internal or proximal to the zucchini genomic sequence encoding the corresponding 6-amino acid hypervariable region of a DHS1 gene in this species: DENITE (SEQ ID NO:137).

The first step will be to sequence the relevant region of the DHS1 gene in the actual zucchini cultivar used for genome editing to confirm the absence of single nucleotide polymorphisms (SNPs) which would affect the annealing efficiency of a gRNA. The nucleic acid sequence for the 6-amino acid hypervariable region within the Cucurbita pepo DHS1 gene sequence is GATGAGAATATAACAGAA (SEQ ID NO:138) and will be used, along with flanking genomic sequence, to generate candidate gRNA sequences. An example of one alternative nucleic acid sequence to generate gRNA is the coding strand gRNA sequence (PAM sequence underlined) TATAACAGAAGATTGCTCTGAGG (SEQ ID NO:139).

Stable transformation of zucchini tissue will be performed with the appropriate selectable and/or scorable markers after plasmid construction, sequence confirmation, and transfer into Agrobacterium vector and host. Healthy callus tissues will be shooted and rooted using the appropriate hormone-enhanced solid media, and T0 zucchini plants will be regenerated and their progeny genotyped for the targeted mutations in the DHS1 gene by Next Generation Sequencing (NGS) or Sanger sequencing of PCR-amplified target regions using flanking primers.

In one embodiment, the T1 plants with confirmed zucchini DHS1 sequence deletions will be selfed, and the T2 plants will be screened for the presence of homozygous DHS1 mutations and absence of transgenes, e.g., null segregants. The selected T2 plants will be selfed and T3 seeds will be propagated to test for increased shelf life, higher yields, and/or greater abiotic/biotic stress tolerance compared to wild-type controls.

### Example 13: Genome Editing of the DHS1 Loci in Potato (Solanum tuberosum)

Guide RNAs (gRNAs) will be designed to anneal with a chosen site in the potato genome and allowed to interact with one or more Cas9 or other CRISPR double-stranded nuclease proteins. These gRNAs will be cloned in a vector and operably-linked to a promoter, e.g., the gRNA cassette, in a plant cell. One or more genes encoding a Cas9 or other CRISPR double-stranded nuclease protein will be cloned in a vector and operably linked to a promoter, e.g., the CRISPR nuclease cassette, in a plant cell (the CRISPR nuclease cassette). The gRNA and CRISPR nuclease cassettes will be cloned into a vector that is suitable for plant transformation, and this vector will be subsequently transformed into Agrobacterium cells. These cells will be brought into contact with potato tissue that is suitable for transformation. Following incubation with Agrobacterium cells, the potato cells will be cultured on a tissue culture medium that is suitable for regeneration of intact plants with selection against Agrobacterium cells. Potato plants will be regenerated from the cells that were brought into contact with Agrobacterium cells harboring the vector that contained the gRNA and CRISPR nuclease cassettes. Following regeneration of the potato plants, DNA will be extracted from harvested plant tissue. DNA sequencing assays will be performed to determine whether a change has occurred in the DNA sequence at the targeted genomic location.

Plasmid construction, genotyping, and plant propagation. As described in Example 5, at least one gRNA will be designed to be internal or proximal to the potato genomic sequence encoding the corresponding 6-amino acid hypervariable region of a DHS1 gene in this species: HELLME (SEQ ID NO:140).

The first step will be to sequence the relevant region of the DHS1 gene in the actual potato cultivar used for genome editing to confirm the absence of single nucleotide polymorphisms (SNPs) which would affect the annealing efficiency of a gRNA. The nucleic acid sequence for the 6-amino acid hypervariable region within the Solanum tuberosum DHS1 gene sequence is CATGAGCTGCTCATGGAG (SEQ ID NO:141) and will be used, along with flanking genomic sequence, to generate candidate gRNA sequences. Examples of two alternative nucleic acid sequences to generate gRNA are the coding strand gRNA sequences (PAM sequences underlined) TTCACATGAGCTGCTCATGGAGG (SEQ ID NO:142) and GCTTTCACATGAGCTGCTCATGG (SEQ ID NO:143).

Stable transformation of potato tissue will be performed with the appropriate selectable and/or scorable markers after plasmid construction, sequence confirmation, and transfer into Agrobacterium vector and host. Healthy callus tissues will be shooted and rooted using the appropriate hormone-enhanced solid media, and T0 potato plants will be regenerated and their progeny genotyped for the targeted mutations in the DHS1 gene by Next Generation Sequencing (NGS) or Sanger sequencing of PCR-amplified target regions using flanking primers.

In one embodiment, the T1 plants with confirmed potato DHS1 sequence deletions will be selfed, and the T2 plants will be screened for the presence of homozygous DHS1 mutations and absence of transgenes, e.g., null segregants. The selected T2 plants will be selfed and T3 seeds will be propagated to test for increased shelf life, higher yields, and/or greater abiotic/biotic stress tolerance compared to wild-type controls.

### Example 14: Genome Editing of the DHS1 Loci in Rice (Oryza sativa Japonica)

Guide RNAs (gRNAs) will be designed to anneal with a chosen site in the rice genome and allowed to interact with one or more Cas9 or other CRISPR double-stranded nuclease proteins. These gRNAs will be cloned in a vector and operably-linked to a promoter, e.g., the gRNA cassette, in a plant cell. One or more genes encoding a Cas9 or other CRISPR double-stranded nuclease protein will be cloned in a vector and operably linked to a promoter, e.g., the CRISPR nuclease cassette, in a plant cell (the CRISPR nuclease cassette). The gRNA and CRISPR nuclease cassettes will be cloned into a vector that is suitable for plant transformation, and this vector will be subsequently transformed into Agrobacterium cells. These cells will be brought into contact with rice tissue that is suitable for transformation. Following incubation with Agrobacterium cells, the rice cells will be cultured on a tissue culture medium that is suitable for regeneration of intact plants with selection against Agrobacterium cells. Rice plants will be regenerated from the cells that were brought into contact with Agrobacterium cells harboring the vector that contained the gRNA and CRISPR nuclease cassettes. Following regeneration of the rice plants, DNA will be extracted from harvested plant tissue. DNA sequencing assays will be performed to determine whether a change has occurred in the DNA sequence at the targeted genomic location.

Plasmid construction, genotyping, and plant propagation. As described in Example 5, at least one gRNA will be designed to be internal or proximal to the rice genomic sequence encoding the corresponding 6-amino acid hypervariable region of a DHS1 gene in this species: HEKPRE (SEQ ID NO:144).

The first step will be to sequence the relevant region of the DHS1 gene in the actual rice cultivar used for genome editing to confirm the absence of single nucleotide polymorphisms (SNPs) which would affect the annealing efficiency of a gRNA. The nucleic acid sequence for the 6-amino acid hypervariable region within the Oryza sativa Japonica DHS1 gene sequence is CACGAGAAGCCACGTGAG (SEQ ID NO:145) and will be used, along with flanking genomic sequence, to generate candidate gRNA sequences. An example of nucleic acid sequence to generate gRNAs in the coding strand (PAM sequence underlined) is: GTCTCACGAGAAGCCACGTGAGG (SEQ ID NO:146). An example of one alternative nucleic acid sequence to generate gRNA is the non-coding strand gRNA sequence (PAM sequence underlined) is GGTCTACAATCTAACCTCCGACA (SEQ ID NO:147).

Stable transformation of rice tissue will be performed with the appropriate selectable and/or scorable markers after plasmid construction, sequence confirmation, and transfer into Agrobacterium vector and host. Healthy callus tissues will be shooted and rooted using the appropriate hormone-enhanced solid media, and T0 rice plants will be regenerated and their progeny genotyped for the targeted mutations in the DHS1 gene by Next Generation Sequencing (NGS) or Sanger sequencing of PCR-amplified target regions using flanking primers.

In one embodiment, the T1 plants with confirmed rice DHS1 sequence deletions will be selfed, and the T2 plants will be screened for the presence of homozygous DHS1 mutations and absence of transgenes, e.g., null segregants. The selected T2 plants will be selfed and T3 seeds will be propagated to test for increased shelf life, higher yields, and/or greater abiotic/biotic stress tolerance compared to wild-type controls.

### Example 15: Genome Editing of the DHS1 Loci in Sorghum (Sorghum bicolor)

Guide RNAs (gRNAs) will be designed to anneal with a chosen site in the sorghum genome and allowed to interact with one or more Cas9 or other CRISPR double-stranded nuclease proteins. These gRNAs will be cloned in a vector and operably-linked to a promoter, e.g., the gRNA cassette, in a plant cell. One or more genes encoding a Cas9 or other CRISPR double-stranded nuclease protein will be cloned in a vector and operably linked to a promoter, e.g., the CRISPR nuclease cassette, in a plant cell (the CRISPR nuclease cassette). The gRNA and CRISPR nuclease cassettes will be cloned into a vector that is suitable for plant transformation, and this vector will be subsequently transformed into Agrobacterium cells. These cells will be brought into contact with sorghum tissue that is suitable for transformation. Following incubation with Agrobacterium cells, the sorghum cells will be cultured on a tissue culture medium that is suitable for regeneration of intact plants with selection against Agrobacterium cells. Sorghum plants will be regenerated from the cells that were brought into contact with Agrobacterium cells harboring the vector that contained the gRNA and CRISPR nuclease cassettes. Following regeneration of the sorghum plants, DNA will be extracted from harvested plant tissue. DNA sequencing assays will be performed to determine whether a change has occurred in the DNA sequence at the targeted genomic location.

Plasmid construction, genotyping, and plant propagation. As described in Example 5, at least one gRNA will be designed to be internal or proximal to the sorghum genomic sequence encoding the corresponding 6-amino acid hypervariable region of a DHS1 gene in this species: HEKPSE (SEQ ID NO:148).

The first step will be to sequence the relevant region of the DHS1 gene in the actual sorghum cultivar used for genome editing to confirm the absence of single nucleotide polymorphisms (SNPs) which would affect the annealing efficiency of a gRNA. The nucleic acid sequence for the 6-amino acid hypervariable region within the Sorghum bicolor DHS1 gene sequence is CATGAGAAGCCCAGTGAG (SEQ ID NO:149) and will be used, along with flanking genomic sequence, to generate candidate gRNA sequences. Examples of two alternative nucleic acid sequences to generate gRNA are the coding strand gRNA sequence (PAM sequences underlined) ATCTCATGAGAAGCCCAGTGAGG (SEQ ID NO:150) and the non-coding strand gRNA sequence GGGTCACTCCTAACACTACTGCG (SEQ ID NO:151).

Stable transformation of sorghum tissue will be performed with the appropriate selectable and/or scorable markers after plasmid construction, sequence confirmation, and transfer into Agrobacterium vector and host. Healthy callus tissues will be shooted and rooted using the appropriate hormone-enhanced solid media, and T0 sorghum plants will be regenerated and their progeny genotyped for the targeted mutations in the DHS1 gene by Next Generation Sequencing (NGS) or Sanger sequencing of PCR-amplified target regions using flanking primers.

In one embodiment, the T1 plants with confirmed sorghum DHS1 sequence deletions will be selfed, and the T2 plants will be screened for the presence of homozygous DHS1 mutations and absence of transgenes, e.g., null segregants. The selected T2 plants will be selfed and T3 seeds will be propagated to test for increased shelf life, higher yields, and/or greater abiotic/biotic stress tolerance compared to wild-type controls.

### Example 16: Genome Editing of the DHS1 Loci in Rose (Rosa chinensis)

Guide RNAs (gRNAs) will be designed to anneal with a chosen site in the rose genome and allowed to interact with one or more Cas9 or other CRISPR double-stranded nuclease proteins. These gRNAs will be cloned in a vector and operably-linked to a promoter, e.g., the gRNA cassette, in a plant cell. One or more genes encoding a Cas9 or other CRISPR double-stranded nuclease protein will be cloned in a vector and operably linked to a promoter, e.g., the CRISPR nuclease cassette, in a plant cell (the CRISPR nuclease cassette). The gRNA and CRISPR nuclease cassettes will be cloned into a vector that is suitable for plant transformation, and this vector will be subsequently transformed into Agrobacterium cells. These cells will be brought into contact with rose tissue that is suitable for transformation. Following incubation with Agrobacterium cells, the rose cells will be cultured on a tissue culture medium that is suitable for regeneration of intact plants with selection against Agrobacterium cells. Rose plants will be regenerated from the cells that were brought into contact with Agrobacterium cells harboring the vector that contained the gRNA and CRISPR nuclease cassettes. Following regeneration of the rose plants, DNA will be extracted from harvested plant tissue. DNA sequencing assays will be performed to determine whether a change has occurred in the DNA sequence at the targeted genomic location.

Plasmid construction, genotyping, and plant propagation. As described in Example 5, at least one gRNA will be designed to be internal or proximal to the rose genomic sequence encoding the corresponding 6-amino acid hypervariable region of a DHS1 gene in this species: DEAVAE (SEQ ID NO:152).

The first step will be to sequence the relevant region of the DHS1 gene in the actual rose cultivar used for genome editing to confirm the absence of single nucleotide polymorphisms (SNPs) which would affect the annealing efficiency of a gRNA. The nucleic acid sequence for the 6-amino acid hypervariable region within the Rosa chinensis DHS1 gene sequence is GATGAGGCTGTAGCTGAG (SEQ ID NO:153) and will be used, along with flanking genomic sequence, to generate candidate gRNA sequences. Two examples of nucleic acid sequences to generate gRNAs in the coding strand (PAM sequence underlined) are: GCGGTGAGGAGGAGAGGGATGGG (SEQ ID NO:154) and GAGGCTGTAGCTGAGGATTGCGG (SEQ ID NO:155).

Stable transformation of rose tissue will be performed with the appropriate selectable and/or scorable markers after plasmid construction, sequence confirmation, and transfer into Agrobacterium vector and host. Healthy callus tissues will be shooted and rooted using the appropriate hormone-enhanced solid media, and T0 rose plants will be regenerated and their progeny genotyped for the targeted mutations in the DHS1 gene by Next Generation Sequencing (NGS) or Sanger sequencing of PCR-amplified target regions using flanking primers.

In one embodiment, the T1 plants with confirmed rose DHS1 sequence deletions will be selfed, and the T2 plants will be screened for the presence of homozygous DHS1 mutations and absence of transgenes, e.g., null segregants. The selected T2 plants will be selfed and T3 seeds will be propagated to test for increased shelf life, higher yields, and/or greater abiotic/biotic stress tolerance compared to wild-type controls.

1. A method of producing a plant with delayed senescence relative to a wild-type control plant, the method comprising inducing at least one nucleotide substitution into at least one copy of a gene encoding deoxyhypusine synthase (DHS) in the plant in a codon for at least one amino acid selected from the group consisting of H76, E77, L78, P79, T80, E81 of SEQ ID NO:106 or a corresponding amino acid in another plant species, wherein the nucleotide substitution decreases the activity of DHS encoded by the gene in the plant relative to the activity of DHS in the wild-type control plant so as to delay senescence.

2. The method of claim 1, wherein the delayed senescence
a) increases seed yield in the plant relative to a wild-type control plant,
b) increases leaf and root biomass relative to a wild-type control plant,
c) enhances plant survival during drought or nutrient stress relative to a wild-type control plant,
d) increases disease resistance of the plant relative to a wild-type control plant; and/or
e) increases the period of time during which leaves, stems, seeds and fruit of the plant may be stored and remain suitable for use relative to a wild-type control plant.

3. The method of claim 1, wherein the plant is a haploid, diploid, or polyploid.

4. The method of claim 1 comprising inducing at least one nucleotide substitution into at least two copies of a gene encoding DHS in the plant.

5. The method of claim 1, wherein the senescence is age-related senescence.

6. The method of claim 1, wherein the senescence is environmental stress-induced senescence.

7. The method of claim 1, wherein the senescence is plant pathogen-induced senescence.

8. A plant produced by the method of claim 1.

9. Progeny of the plant according to claim 1, wherein the progeny comprises the nucleotide substitution.

10. The method of claim 1, wherein the plant is selected from the group consisting of Arachis hypogaea, Beta vulgaris, Brassica napus, Brassica rapa, Camelina sativa, Camellia sinensis, Cannabis sativa, Capsicum anuum, Cicer arietinum, Coffea canephora, Cucurbita pepo, Fragaria ananassa, Glycine max, Gossypium hirsutum, Lactuca sativa, Manihot esculenta, Medicago sativa, Mentha longifolia, Musa acuminate, Oryza sativa, Phalaenopsis equestris, Phaseolus vulgaris, Populus deltoides, Rosa chinensis, Solanum lycopersicum, Solanum tuberosum, Sorghum bicolor, Theobroma cacao, Triticum aestivum, Vitis labrusca, Vitis vinifera, Zea mays.

11. A method of producing a plant with delayed senescence relative to a wild-type control plant, the method comprising inducing at least one nucleotide deletion or insertion into at least one copy of a gene encoding deoxyhypusine synthase (DHS) in the plant in a codon for at least one amino acid selected from the group consisting of H76, E77, L78, P79, T80, E81 of SEQ ID NO:106, or a corresponding amino acid in another plant species, wherein the nucleotide deletion or insertion decreases the activity of DHS encoded by the gene in the plant relative to the activity of DHS in the wild-type control plant so as to delay senescence, and the plant contains at least one copy of the gene encoding DHS without the deletion or insertion.

12. The method of claim 0, wherein the delayed senescence
a) increases seed yield in the plant relative to a wild-type control plant,
b) increases leaf and root biomass relative to a wild-type control plant,
c) enhances plant survival during drought or nutrient stress relative to a wild-type control plant,
d) increases disease resistance of the plant relative to a wild-type control plant, and/or
e) increases the period of time during which leaves, stems, seeds and fruit of the plant may be stored and remain suitable for use relative to a wild-type control plant.

13. The method of claim 11, wherein the plant is haploid, diploid, or polyploid.

14. The method of claim 11, wherein the senescence is age-related senescence.

15. The method of claim 11, wherein the senescence is environmental stress-induced senescence.

16. The method of claim 11, wherein the senescence is plant pathogen-induced senescence.

17. A plant produced by the method of claim 11.

18. Progeny of the plant according to claim 11, wherein the progeny comprises the nucleotide deletion or insertion.

19. The method of claim 11, wherein the plant is selected from the group consisting of Arachis hypogaea, Beta vulgaris, Brassica napus, Brassica rapa, Camelina sativa, Camellia sinensis, Cannabis sativa, Capsicum anuum, Cicer arietinum, Coffea canephora, Cucurbita pepo, Fragaria ananassa, Glycine max, Gossypium hirsutum, Lactuca sativa, Manihot esculenta, Medicago sativa, Mentha longifolia, Musa acuminate, Oryza sativa, Phalaenopsis equestris, Phaseolus vulgaris, Populus deltoides, Rosa chinensis, Solanum lycopersicum, Solanum tuberosum, Sorghum bicolor, Theobroma cacao, Triticum aestivum, Vitis labrusca, Vitis vinifera, Zea mays.
